SEQUENCE LISTING (56 sequences)

SEQ ID NO: 1 - DNA, 56 bp, Artificial Sequence
  Synthetic DNA Sequence
acaacacattttactgctgtgaactttgttgtaatcctgcctgtgctggatgttat

SEQ ID NO: 2 - protein, 19 aa, Artificial Sequence
  Synthetic Peptide
NNTFYCCELCCNPACAGCY

SEQ ID NO: 3 - protein, 18 aa, Artificial Sequence
  Synthetic Peptide
NTFYCCELCCYPACAGCN

SEQ ID NO: 4 - DNA, 56 bp, Artificial Sequence
  Synthetic DNA Sequence
atagtagcaattactgctgtgaattgtgttgtaatcctgcttgtaacgggtgctat

SEQ ID NO: 5 - protein, 19 aa, Artificial Sequence
  Synthetic Peptide
NSSNYCCELCCNPACNGCY

SEQ ID NO: 6 - protein, 15 aa, Artificial Sequence
  Synthetic Peptide
PNTCEICAYAACTGC

SEQ ID NO: 7 - protein, 18 aa, Artificial Sequence
  Synthetic Peptide
NNTFYCCELCCNPACAGC

SEQ ID NO: 8 - protein, 17 aa, Artificial Sequence
  Synthetic Peptide
NTFYCCELCCNPACAGC

SEQ ID NO: 9 - protein, 16 aa, Artificial Sequence
  Synthetic Peptide
TFYCCELCCNPACAGC

SEQ ID NO: 10 - protein, 15 aa, Artificial Sequence
  Synthetic Peptide
FYCCELCCNPACAGC

SEQ ID NO: 11 - protein, 14 aa, Artificial Sequence
  Synthetic Peptide
YCCELCCNPACAGC

SEQ ID NO: 12 - protein, 13 aa, Artificial Sequence
  Synthetic Peptide
CCELCCNPACAGC

SEQ ID NO: 13 - protein, 18 aa, Artificial Sequence
  Synthetic Peptide
NTFYCCELCCNPACAGCY

SEQ ID NO: 14 - protein, 17 aa, Artificial Sequence
  Synthetic Peptide
TFYCCELCCNPACAGCY

SEQ ID NO: 15 - protein, 16 aa, Artificial Sequence
  Synthetic Peptide
FYCCELCCNPACAGCY

SEQ ID NO: 16 - protein, 15 aa, Artificial Sequence
  Synthetic Peptide
YCCELCCNPACAGCY

SEQ ID NO: 17 - protein, 14 aa, Artificial Sequence
  Synthetic Peptide
CCELCCNPACAGCY

SEQ ID NO: 18 - protein, 17 aa, Artificial Sequence
  Synthetic Peptide
NTFYCCELCCYPACAGC

SEQ ID NO: 19 - protein, 16 aa, Artificial Sequence
  Synthetic Peptide
TFYCCELCCYPACAGC

SEQ ID NO: 20 - protein, 15 aa, Artificial Sequence
  Synthetic Peptide
FYCCELCCYPACAGC

SEQ ID NO: 21 - protein, 14 aa, Artificial Sequence
  Synthetic Peptide
YCCELCCYPACAGC

SEQ ID NO: 22 - protein, 13 aa, Artificial Sequence
  Synthetic Peptide
CCELCCYPACAGC

SEQ ID NO: 23 - protein, 17 aa, Artificial Sequence
  Synthetic Peptide
TFYCCELCCYPACAGCN

SEQ ID NO: 24 - protein, 16 aa, Artificial Sequence
  Synthetic Peptide
FYCCELCCYPACAGCN

SEQ ID NO: 25 - protein, 15 aa, Artificial Sequence
  Synthetic Peptide
YCCELCCYPACAGCN

SEQ ID NO: 26 - protein, 14 aa, Artificial Sequence
  Synthetic Peptide
CCELCCYPACAGCN

SEQ ID NO: 27 - protein, 18 aa, Artificial Sequence
  Synthetic Peptide
NSSNYCCELCCNPACTGC

SEQ ID NO: 28 - protein, 17 aa, Artificial Sequence
  Synthetic Peptide
SSNYCCELCCNPACTGC

SEQ ID NO: 29 - protein, 16 aa, Artificial Sequence
  Synthetic Peptide
SNYCCELCCNPACTGC

SEQ ID NO: 30 - protein, 15 aa, Artificial Sequence
  Synthetic Peptide
NYCCELCCNPACTGC

SEQ ID NO: 31 - protein, 14 aa, Artificial Sequence
  Synthetic Peptide
YCCELCCNPACTGC

SEQ ID NO: 32 - protein, 13 aa, Artificial Sequence
  Synthetic Peptide
CCELCCNPACTGC

SEQ ID NO: 33 - protein, 18 aa, Artificial Sequence
  Synthetic Peptide
SSNYCCELCCNPACTGCY

SEQ ID NO: 34 - protein, 17 aa, Artificial Sequence
  Synthetic Peptide
SNYCCELCCNPACTGCY

SEQ ID NO: 35 - protein, 16 aa, Artificial Sequence
  Synthetic Peptide
NYCCELCCNPACTGCY

SEQ ID NO: 36 - protein, 15 aa, Artificial Sequence
  Synthetic Peptide
YCCELCCNPACTGCY

SEQ ID NO: 37 - protein, 14 aa, Artificial Sequence
  Synthetic Peptide
CCELCCNPACTGCY

SEQ ID NO: 38 - protein, 18 aa, Artificial Sequence
  Synthetic Peptide
NTFYCCELCCNPACAGCY

SEQ ID NO: 39 - protein, 18 aa, Artificial Sequence
  Synthetic Peptide
NTFYCCELCCAPACAGCY

SEQ ID NO: 40 - protein, 18 aa, Artificial Sequence
  Synthetic Peptide
NTFYCCELCCNAACAGCY

SEQ ID NO: 41 - protein, 17 aa, Artificial Sequence
  Synthetic Peptide
NTFYCCELCCNPACAGC

SEQ ID NO: 42 - protein, 15 aa, Artificial Sequence
  Synthetic Peptide
YCCELCCNPACAGCY

SEQ ID NO: 43 - protein, 14 aa, Artificial Sequence
  Synthetic Peptide
YCCELCCNPACAGC

SEQ ID NO: 44 - protein, 14 aa, Artificial Sequence
  Synthetic Peptide
CCELCCNPACAGCY

SEQ ID NO: 45 - protein, 13 aa, Artificial Sequence
  Synthetic Peptide
CCELCCNPACAGC

SEQ ID NO: 46 - protein, 25 aa, Artificial Sequence
  Synthetic Peptide
QACDPPSPPAEVCCDVCCNPACAGC

SEQ ID NO: 47 - protein, 16 aa, Artificial Sequence
  Synthetic Peptide
IDCCICCNPACFGCLN

SEQ ID NO: 48 - protein, 18 aa, Artificial Sequence
  Synthetic Peptide
SSDWDCCDVCCNPACAGC

SEQ ID NO: 49 - protein, 19 aa, Artificial Sequence
  Synthetic Peptide
NSSNYCCELCCYPACTGCY

SEQ ID NO: 50 - protein, 13 aa, Artificial Sequence
  Synthetic Peptide
CCDVCCNPACTGC

SEQ ID NO: 51 - protein, 14 aa, Artificial Sequence
  Synthetic Peptide
CCDVCCYPACTGCY

SEQ ID NO: 52 - protein, 14 aa, Artificial Sequence
  Synthetic Peptide
CCDLCCNPACAGCY

SEQ ID NO: 53 - protein, 14 aa, Artificial Sequence
  Synthetic Peptide
CCQLCCNPACTGCY

SEQ ID NO: 54 - protein, 15 aa, Artificial Sequence
  Synthetic Peptide
PGTCEICAYAACTGC

SEQ ID NO: 55 - protein, 15 aa, Artificial Sequence
  Synthetic Peptide
QEDCELCINVACTGC

SEQ ID NO: 56 - protein, 16 aa, Artificial Sequence
  Synthetic Peptide
NDDCELCVNVACTGCL

(19) United States
(12) Patent Application Publication
(10) Pub. No.: US 2014/0255518 A1
(43) Pub. Date: Sep. 11, 2014

(54) Treatment and Prevention of Gastrointestinal Syndrome

(71) Applicants: Peng Li, Salt Lake City, UT (US); Scott A. Waldman, Ardmore, PA (US); Evan Wuthrick, Philadelphia, PA (US)

(72) Inventors: Peng Li, Salt Lake City, UT (US); Scott A. Waldman, Ardmore, PA (US); Evan Wuthrick, Philadelphia, PA (US)

(73) Assignees: THOMAS JEFFERSON UNIVERSITY HOSPITALS, INC., Philadelphia, PA (US); THOMAS JEFFERSON UNIVERSITY, Philadelphia, PA (US)

(21) Appl. No.: 14/114,727

(22) PCT Filed: Apr. 27, 2012
(86) PCT No.: PCT/US2012/035376

§ 371 Date: Apr. 25, 2014

Related U.S. Application Data
(60) Provisional application No. 61/480,792, filed on Apr. 29, 2011.

(51) Int. Cl. C07K 14/58; C07K 14/575; A61K 45/06; A61K 38/22

(52) U.S. Cl. CPC C07K 14/58; A61K 38/2242; A61K 38/22; C07K 14/575; A61K 45/06
USPC 424/718; 514/12.4

(57) ABSTRACT

Compositions comprising a guanylyl cyclase C agonist in an amount effective to protect intestinal tissue against radiation or chemotherapy and methods of using such compositions to prevent GI syndrome in cancer patient undergoing radiation or chemotherapy and in individuals exposed to or susceptible to exposure to radiation are disclosed.

### FIELD OF THE INVENTION

The present invention relates to compositions for and methods of protecting an individual from serious and possibly lethal effects associated with exposure to radiation and some toxic compounds. The present invention relates to compositions for and methods of protecting an individual from serious and possibly lethal side effects associated with cancer chemotherapy and radiation therapy. The compositions and methods are particularly useful to protect the gastrointestinal (GI) tract from GI syndrome caused by radiation.

### BACKGROUND OF THE INVENTION

Whether delivered intentionally as part of a treatment regimen to a cancer patient or as the result of a catastrophic event which results in the deliberate or accidental release of radiation among a population, exposure to high levels of ionizing radiation is toxic and can be lethal. The gastrointestinal (GI) track, with its large number of dividing cells, is particularly susceptible to deleterious effects of radiation. The GI syndrome induced by radiation includes severe diarrhea, fever, dehydration, and imbalance in the electrolytes (sodium, potassium, etc). In cases of high levels of exposure, the results can be lethal. Death may occur within 2 weeks of exposure.

Cancer is a leading cause of death worldwide: it accounted for 7-8 million deaths (approximately 13% of all deaths) yearly since 2004. Deaths from cancer worldwide are projected to continue rising, with an estimated 12 million deaths in 2030. Lung, stomach, liver, colon and breast cancer cause the most cancer deaths each year. In US, cancer is the second cause of death in adults and causes above half a million deaths each year. Lung, prostate, breast and colon cancers are the leading causes of cancer related deaths.

Chemotherapy and radiation therapy, the two most common types of cancer treatment, work by destroying fast-growing cells such as cancer cells. Chemotherapy and radiation therapy are extremely toxic treatments because they target rapidly dividing cells. Therefore, as an unwanted side effect of chemotherapy and radiation, other types of fast-growing normal cells in the body, such as hematopoietic, hair and gastrointestinal tract (GI) cells, are also damaged and killed. Severe side effects of chemo- and radiation therapy discourage people from continuing their therapy, limit the efficacy of the treatments and sometimes even kill patients. The toxicity which is manifested by these side effects limits the dosages of chemotherapeutic and radiation a patient can be administered.

Gastrointestinal toxicities occur in clinical practice as a side effect of treatment with radiation and some chemotherapeutic agents. Additionally, a 1-3% treatment related death rate has been observed in this and many other large phase III clinical trials. While side effects can be lethal, most acute side effects improve over time. Some chronic side effects of cancer treatment, however, can lead to lifelong morbidity. Minimizing the side effects of chemotherapy and radiation remains one of the top priorities for patients and doctors like.

Mice irradiated with >15 Gy of radiation die between 7 and 12 days after treatment from complications of damage to the small intestine—gastrointestinal (GI) syndrome—prior to development of lethal effect of hemopoietic cells. Massive p53-dependent apoptosis is observed following lethal doses of radiation, suggesting that p53 is a determinant of radiation-induced death. However, while the reaction of small intestine to gamma radiation has been well examined at a pathomorphological level, the exact cause of GI lethality has not been fully eludicated. Death may occur as a direct consequence of the damage of epithelial crypt cells and followed denudation of villi leading to fluid and electrolyte imbalance, bacteremia and endotoxemia. Besides inflammation and stromal responses, endothelial dysfunctions may also contribute to lethality.

Garin-Laflam, et al. Am. J. Physiol Gastrointest Liver Physiol 2009 296 G740-9, show the involvement of GCC and cGMP in the prevention of radiation induced intestinal epithelial apoptosis. These studies which relate relative number of intestinal cells undergoing apoptosis, not survival from GI syndrome, were conducted to resolve whether GCC activation has a pro-apoptotic effect, an anti-apoptotic effect or neither in a model of apoptosis involving cells that express GCC. In these studies, intestinal tissue was removed from mice and the number of cells in the resected tissue undergoing apoptosis was measured. Tissue was obtained from various wild type and genetically modified mice as well as mice injected with a cGMP analog. The experiments showed that tissue removed from irradiated mice included a larger number of cells undergoing apoptosis compared to levels observed in tissue from non-irradiated animals. Further, the data show tissue removed from irradiated mice that lacked genes encoding GCC or uroguanylin included a larger number of cells undergoing apoptosis compared to levels observed in tissue from irradiated wild type mice. Experiments also showed cGMP supplementation ameriolated the level of apoptosis in irradiated intestinal tissue of mice lacking genes encoding GCC or uroguanylin but not in wild type mice.

Hendry et al. Radiation Research 1997148(3):254-9 report that radiation induced apoptosis of intestinal cells does not correlate with the survival rate of clonogenic cells responsible for the recovery of epithelial cells of the intestine.

Komarova et al. Oncogene (2004) 23, 3265-3271 use p53 deficient mice to show that cell cycle arrest following irradiation prolongs survival by delaying crypt cells from entry into a mitotic catastrophe and fast death after being damaged by radiation. Arresting proliferation of crypt cells after irradiation enhances survival of epithelium of the small intestine. The cycle arrest is attributed to a protective role of p53 through its growth arrest rather than apoptotic function.

Kirsch et al, Science 2010 327:593-6 report that radiation induced gastrointestinal syndrome is apoptosis independent. Using genetically modified mice which have tissue specific suppression of apoptosis essential genes, the authors show that radiation induced gastrointestinal syndrome can proceed in the absence of a complete compliment of proteins required to undergo apoptosis, and therefore that radiation induced gastrointestinal syndrome is independent of the intrinsic apoptosis pathway. Deletion of p53 expression in epithelial cells sensitized irradiated mice to radiation induced gastrointestinal syndrome while overexpression of p53 was protective. The data show that p53 expression is linked to survival following high doses of ionizing radiation even in animals which lack other proteins essential to the intrinsic apoptosis pathway; radiation induced gastrointestinal syndrome is independent of apoptosis.

There remains a need for treatments which minimize the side effects chemotherapy and radiation therapy in order to increase patient comfort and to allow for an increase in dosage which would otherwise be prevented due to unacceptable levels of side effects. Potentiating the therapeutic efficacy for cancer treatment by prevention of the side effects of chemotherapy and radiation therapy and increasing susceptibility to cancer cells represents a major advance in the treatment of cancer. There remains a need to identify compositions and methods of preventing GI syndrome and reducing the severity of gastrointestinal side effects following exposure to toxic chemotherapy or radiation. There remains a need to protect gastrointestinal cells from damage by exposure to toxic chemotherapy or radiation leading to GI syndrome. There remains a need to reduce lethal effects of radiation and chemotherapy due to damage to gastrointestinal cells and increasing the tolerable levels of toxic chemotherapy and radiation in order to provide more effective therapy.

### SUMMARY OF THE INVENTION

The present invention also relates to compositions comprising a guanylyl cyclase C agonist in an amount effective to protect intestinal tissue against radiation or chemotherapy.

The present invention relates to methods of preventing GI syndrome and reducing side effects in cancer patient undergoing radiation or chemotherapy.

The present invention relates to methods of preventing GI syndrome in individuals exposed to or susceptible to exposure to radiation.

Some embodiments of the invention relates to methods of preventing GI syndrome in individuals undergoing chemotherapy or radiation therapy to treat cancer. The methods comprise the step of, prior to administration of chemotherapy or radiation to the individual, administering to the individual an amount of one or more compounds that elevates intracellular cGMP levels in gastrointestinal cells sufficient to arrest cell proliferation of gastrointestinal cells and/or maintain genomic integrity by enhanced DNA damage sensing and repair for a period sufficient to prevent GI syndrome.

Some embodiments of the invention relate to methods of reducing gastrointestinal side effects in individuals undergoing chemotherapy or radiation therapy to treat cancer. The methods comprise the steps of, prior to administration of chemotherapy or radiation to the individual, administering to the individual an amount of one or more compounds that elevates intracellular cGMP levels in gastrointestinal cells sufficient to arrest cell proliferation of gastrointestinal cells and/or maintain genomic integrity by enhanced DNA damage sensing and repair for a period sufficient to increase survival of gastrointestinal cells and reduce severity of chemotherapy or radiation therapy side effects.

Some embodiments of the invention relate to methods of treating individuals who have cancer. The methods comprising the steps of administering to the individual an amount of one or more compounds that elevates intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome; and then administering to the individual chemotherapy or radiation an amount sufficient to treat cancer.

Some embodiments of the invention relate to methods of treating individuals who have cancer. The methods comprise the steps of administering to the individual an amount of one or more compounds that elevates intracellular cGMP levels in gastrointestinal cells sufficient to arrest cell proliferation of said gastrointestinal cells and/or maintain genomic integrity by enhanced DNA damage sensing and repair for a period sufficient to increase survival of gastrointestinal cells and reduce severity of chemotherapy or radiation therapy side effects; and then administering to the individual chemotherapy or radiation an amount sufficient to treat cancer.

Some embodiments of the invention relate to methods of preventing GI syndrome in individuals undergoing chemotherapy or radiation therapy to treat cancer comprising the step of administering to an individual prior to administration of chemotherapy or radiation a population of bacteria comprising bacteria which comprise a nucleic acid molecule that encodes guanylyl cyclase C agonist operably linked to regulatory sequences operable in the bacteria. The bacteria is of a species that can live in a human colon as part of a human's gut flora and express guanylyl cyclase C agonist in an amount sufficient to elevate intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome.

Some embodiments of the invention relate to methods of reducing gastrointestinal side effects in individuals undergoing chemotherapy or radiation therapy to treat cancer comprising the step of administering to an individual prior to administration of chemotherapy or radiation a population of bacteria comprising bacteria which comprise a nucleic acid molecule that encodes guanylyl cyclase C agonist operably linked to regulatory sequences operable in the bacteria. The bacteria is of a species that can live in a human colon as part of a human's gut flora and express guanylyl cyclase C agonist in an amount sufficient to elevate intracellular cGMP levels in gastrointestinal cells sufficient to arrest cell proliferation of said gastrointestinal cells and/or maintain genomic integrity by enhanced DNA damage sensing and repair for a period sufficient to reducing gastrointestinal side effects.

Some embodiments of the invention relate to compositions comprising a guanylyl cyclase C agonist in an amount effective to prevent GI syndrome in an individual undergoing chemotherapy or radiation therapy by elevating intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome.

Some embodiments of the invention relate to compositions comprising a guanylyl cyclase C agonist in an amount effective to reduce gastrointestinal side effects in an individual undergoing chemotherapy or radiation therapy by elevating intracellular cGMP levels in gastrointestinal cells sufficient to arrest cell proliferation of said gastrointestinal cells and/or maintain genomic integrity by enhanced DNA damage sensing and repair for a period sufficient to reducing gastrointestinal side effects.

Some embodiments of the invention comprise methods of preventing GI syndrome in individuals who have been exposed to or who are at risk of exposure to sufficient doses of radiation to cause GI syndrome. The methods comprise the step of administering to such an individual who has been identified as an individual who has been exposed to or who is at risk of exposure to sufficient doses of radiation to cause GI syndrome, an amount of one or more compounds that elevates cGMP levels in gastrointestinal cells sufficient to prevent GI syndrome.

Some embodiments of the invention comprise methods of treating individuals who have been exposed to a sufficient amount of radiation to cause radiation sickness comprising the step of administering to such an individual, an amount of one or more compounds that elevates cGMP levels in gastrointestinal cells sufficient to elevate intracellular cGMP levels in gastrointestinal cells sufficient to arrest cell proliferation of said gastrointestinal cells and/or maintain genomic integrity by enhanced DNA damage sensing and repair for a period sufficient to reduce gastrointestinal damage.

Some embodiments of the invention relate to methods of preventing side effects in individuals who are undergoing chemotherapy or radiation. The methods comprise the steps of administering to said individual prior to administration of chemotherapy or radiation an amount of one or more compounds that elevates cGMP levels in cells to be protected sufficient to arrest cell proliferation of said cells and/or maintain genomic integrity by enhanced DNA damage sensing and repair for a period sufficient to reduce damage to said cells.

Some embodiments of the invention relate to methods of treating individual who have cancer comprising the steps of administering to an individual who has cancer an amount of one or more compounds that elevates cGMP levels in cells to be protected sufficient to arrest cell proliferation of said cells and/or maintain genomic integrity by enhanced DNA damage sensing and repair for a period sufficient to reduce damage to said cells; and administering to the individual chemotherapy or radiation an amount sufficient to treat cancer.

### BRIEF DESCRIPTION OF THE DRAWINGS

FIG. 1 (A-J) show data from experiments comparing levels off apoptosis directly or by detection of indicators whose expression are linked to apoptosis in intestinal tissue from mice that express GCC or knock out mice lacking GCC.

FIG. 2 (A-G) show data from experiments comparing, in irradiated mice, levels off apoptosis directly or by detection of indicators whose expression are linked to apoptosis in intestinal tissue from mice that express GCC or knock out mice lacking GCC as well as mice which have uroguanylin to those that do not have uroguanylin. Survival data is also shown.

FIG. 3 (A-F) show data from experiments comparing, in cells subjected to genotoxic insult, the ability of cGMP to protect human intestinal epithelial cells from cell death to the ability of cGMP to potentiate cell death in human breast, liver and prostate cancer cells. The role of p53 in cGMP-induced protection is also shown in the data.

### DESCRIPTION OF PREFERRED EMBODIMENTS

### Definitions

As used herein the terms “guanylyl cyclase A agonist” and “GCA agonists” are used interchangeably and refer to molecules which bind to guanylyl cyclase A on a cell surface and thereby induce its activity which results in cGMP accumulation within the cell.

As used herein the terms “guanylyl cyclase B agonist” and “GCB agonists” are used interchangeably and refer to molecules which bind to guanylyl cyclase B on a cell surface and thereby induce its activity which results in cGMP accumulation within the cell.

As used herein the terms “guanylyl cyclase C agonist” and “GCC agonists” are used interchangeably and refer to molecules which bind to guanylyl cyclase C on a cell surface and thereby induce its activity which results in cGMP accumulation within the cell.

As used herein the terms “soluble guanylyl cyclase activator” and “sGC activator” are used interchangeably and refer to molecules which bind to soluble guanylyl cyclase and thereby induce its activity which results in cGMP accumulation within the cell.

As used herein the terms “phosphodiesterase inhibitor” and “PDE inhibitors” are used interchangeably and refer to molecules which inhibit the activity of one or more forms or subtypes of the cGMP-hydrolyzing phosphodiesterase enzyme and thereby bringing about cGMP accumulation within the cell.

As used herein the terms “multidrug resistance-associated protein inhibitors” and “MRP inhibitors” are used interchangeably and refer to molecules which inhibit the activity of one or more forms or subtypes of the cGMP-transporting MRPs and thereby bringing about cGMP accumulation within the cell.

As used herein the term “effective amount” refers to the amount of compound(s) effective to result in the accumulation of intracellular cGMP levels to arrest cell proliferation of gastrointestinal cells and/or maintain genomic integrity by enhanced DNA damage sensing and repair for a period sufficient to reduce cell damage caused by chemotherapy or radiation sufficient to reduce the severity of side effects or prevent GUI syndrome and/or radiation sickness.

cGMP

The intracellular accumulation of cGMP helps the cell maintain genomic integrity by enhanced DNA damage sensing and repair for a period sufficient to reduce cell damage caused by chemotherapy or radiation. The p53 protects irradiated cells from mitotic catastrophe by mediating arrest of cell proliferation to allow repair prior to cell division and thereby preventing cell death by mitotic catastrophe.

Side effects caused by radiation and chemotherapy including GI syndrome can be reduced by p53 mediated cell arrest. Increasing intracellular cGMP levels results in enhanced p53 mediated cell arrest when such cells are exposed to lethal toxic chemotherapy or ionizing radiation insults. Increasing intracellular cGMP may be achieved by increasing its production and/or inhibiting its degradation or expulsion from cells. DNA damage repair may be promoted which in turn prevents the death of normal intestinal epithelial cells in response to chemotherapy and ionizing radiation insults.

Accordingly, in conjunction with administration of chemotherapy or radiation to the individual, individual are administered an amount of one or more compounds that elevates intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome. The one or more compounds that elevates intracellular cGMP levels may be administered prior to and/or simultaneous with and/or subsequent to administration of chemotherapy or radiation to the individual although typically, pretreatment one or more compounds that elevates intracellular cGMP levels is performed to ensure the p53 mediated cell protection is initiated before exposure to toxic chemicals or radiation.

While increases in cGMP levels protect intestinal cells following a toxic insult, cGMP potentiates cell death in other cancer cells such as human breast, liver and prostate cancer. By inducing cGMP levels in intestinal epithelial cells to levels sufficient to maintain p53 mediated cell arrest prior to and in conjunction with administration of chemotherapy or radiation therapy, lethal side effects can be reduced, increased doses of chemotherapy or radiation therapy can be utilized and such therapy may be rendered more effective against cancer. When cGMP levels in intestinal epithelial cells are increased sufficient to result in a protection of such cells from toxins and radiation, chemotherapy and radiation therapy may proceed with reduced side effects and risks, even in some cases at higher doses which could not be tolerated absent the protection afforded by the elevated cGMP levels in the intestinal epithelial cells. Moreover, a simultaneous increase in cGMP in cancer cells in the patient may provide synergistic effects on chemotherapy and radiation therapy. The preconditioning of GI tract and targeted organs with treatments that result in intracellular accumulation of cGMP may dramatically increase the efficacy of chemotherapy or radiation therapy by broadening the therapeutic window and increasing the therapeutic index.

The intracellular increase of cGMP levels enhances p53 mediated cell survival in the intestine thereby limiting side effect of chemotherapy and radiation therapy in cancer patients. Thus, increasing intracellular cGMP levels in intestinal cells in particular can be effected prior to chemotherapy and radiation therapy at a time such that during the time when the patient is undergoing chemotherapy or and radiation therapy, the intestinal cells with are protected by p53 thus reducing typical side effects of chemotherapy and radiation therapy. To protect intestinal epithelial cells during chemotherapy and radiation therapy cGMP levels must be increased to an amount effective to enhance p53 mediated cell survival. Since radiation damage and the GI syndrome which results in severe and sometimes lethal side effects in patients receiving radiation is reduced by p53 and independent of apoptosis, the increased level cGMP levels must be sufficient to enhance p53 mediated cell survival.

On the other hand, an increase in intracellular cGMP may also potentiate cancer cell death in response to genetic insults by chemotherapy or ionizing radiation by promoting cell apoptosis in lung, prostate, breast, colorectal and liver cancer cells. Data suggest that cellular preconditioning with cGMP, or agents that result in increased levels of cGMP, in target organs and in the GI tract potentiate chemotherapy and radiation therapy (kill cancer cells) in the target organs while preventing GI tract (normal intestinal cell) damage.

The use of compounds which increase cGMP productions and/or compounds which inhibit cGMP degradation or export from the cell result in an increase in cGMP levels. When administered to the normal GI tract, the increase in cGMP levels serves to protect the cells from cell death which is associated with side effects associated with chemotherapy and radiation therapy, thereby increasing safety of these therapies. In addition, the reduction of side effects allows for toleration of increasing and more effective doses. When delivered to cancer cells such as lung, breast, prostate, colorectal, and liver cancers in order to increase cGMP levels, the cancer cells may become more susceptible to chemotherapy and radiation therapy thereby increasing the efficacy of the treatment.

Compounds which increase cGMP production include activators of guanylyl cyclases including three cellular receptor forms guanylyl cyclase A (GCA), guanylyl cyclase B (GCB) and guanylyl cyclase C (GCC) as well as soluble guanylyl cyclase (sGC).

Compounds which inhibit cGMP degradation and/or export from the include phosphodiesterase enzyme (PDE) inhibitors which inhibit PDE forms and subtypes involved in converting cGMP.

Compounds which inhibit cGMP export from the cell include multidrug resistance protein (MRP) inhibitors which inhibit MRP forms and subtypes involved in transport of cGMP.

These compounds can be used alone or in combinations of two or more to increase intracellular cGMP levels to protect cells of the intestines from cell death associated with chemotherapy and radiation therapy side effects and may render cancer cells more susceptible to cell death.

### GCC

GCC is the predominant guanylyl cyclase in the GI tract. Accordingly, the use of GCC activators or agonists is particularly effective to increase intracellular cGMP in the GI tract. The GCC activators include endogenous peptides guanylin and uroguanylin as well as heat stable enterotoxins produced by bacteria, such as E. coli STs. PDE inhibitors and MRP inhibitors are also known. In some embodiments, one or more GCC agonists is used. In some embodiments, one or more PDE inhibitors is used. In some embodiments, one or more MRP inhibitors is used. In some embodiments, a combination of one or more GCC agonists and/or one or more PDE inhibitors and/or one or more MRP inhibitors is used.

Activation of the cellular receptor guanylyl cyclase C (GCC), a protein expressed primarily in the GI tract, protects cells in the GI tract from dying in response to toxic chemotherapy or ionizing radiation insults. The activation of GCC leads to intracellular accumulation of cGMP which enhances p53 mediated cell survival. Many side effects caused by radiation and chemotherapy can be reduced by enhancing p53 mediated cell survival. By activating GCC, intracellular cGMP levels are increased resulting in enhanced p53 mediated cell survival when such cells are exposed to lethal toxic chemotherapy or ionizing radiation insults.

GCC is the intestinal epithelial cell receptor for the endogenous paracrine hormones guanylin and uroguanylin. Diarrheagenic bacterial heat-stable enterotoxins (STs) also target GCC. Hormone-receptor interaction between guanylin or uroguanylin and the extracellular domain of GCC or ST-receptor interaction between the peptide enterotoxin ST and the extracellular domain of GCC each activates the intracellular catalytic domain of GCC which converts GTP to cyclic GMP (cGMP). This cyclic nucleotide, as a second messenger, activates its downstream effectors mediating GCC's cellular effects. Increasing intracellular cGMP by activating guanylyl cyclase (including particulate and soluble forms) or by inhibiting cGMP degradation or expulsion by inhibitors of phosphodiesterases (PDEs) or multi-drug resistance associated proteins (MRPs), respectively, promotes DNA damage repair which in turn prevents the death of normal intestinal epithelial cells in response to chemotherapy and ionizing radiation insults.

Increases in cGMP levels such as those increases associated with GCC activation protect intestinal cells through p53 mediated cell survival following a toxic insult. Thus, activation of GCC can be effected prior to chemotherapy and radiation therapy at a time such that during the time when the patient is undergoing chemotherapy or and radiation therapy, the GCC activated intestinal cells are protected from typical side effect of chemotherapy and radiation therapy by p53 mediated cell survival. In addition to activation of GCC, protection of intestinal epithelial cells during chemotherapy and radiation therapy can be undertaken by increasing cGMP levels to an amount effective to enhance p53 mediated cell survival.

Since radiation damage and the GI syndrome which results in severe and sometimes lethal side effects in patients receiving radiation is independent of apoptosis and can be mitigated by p53, the level of GCC activation or other increase in cGMP levels must be sufficient to enhance p53 mediated cell survival.

Administration of a GCC agonist refers to administration of one or more compounds that bind to and activate GCC.

Guanylyl cyclase C (GCC) is a cellular receptor expressed by cells lining the large and small intestines. The binding of GCC agonists to GCC in the gastrointestinal track is known to activate GCC, leading to an increase in intracellular cGMP, which results in activation of downstream signaling events.

### GCC Agonists

GCC agonists are known. Two native GCC agonists, guanylin and uroguanylin, have been identified (see U.S. Pat. Nos. 5,969,097 and 5,489,670, which are each incorporated herein by reference. In addition, several small peptides, which are produced by enteric pathogens, are toxigenic agents which cause diarrhea (see U.S. Pat. No. 5,518,888, which is incorporated herein by reference). The most common pathogen derived GCC agonist is the heat stable enterotoxin produced by strains of pathogenic E. coli. Native heat stable enterotoxin produced by pathogenic E. coli is also referred to as ST. A variety of other pathogenic organisms including Yersinia and Enterobacter, also make enterotoxins which can bind to guanylyl cyclase C in an agonistic manner. In nature, the toxins are generally encoded on a plasmid which can “jump” between different species. Several different toxins have been reported to occur in different species. These toxins all possess significant sequence homology, they all bind to ST receptors and they all activate guanylate cyclase, producing diarrhea.

ST has been both cloned and synthesized by chemical techniques. The cloned or synthetic molecules exhibit binding characteristics which are similar to native ST. Native ST isolated from E. coli is 18 or 19 amino acids in length. The smallest “fragment” of ST which retains activity is the 13 amino acid core peptide extending toward the carboxy terminal from cysteine 6 to cysteine 18 (of the 19 amino acid form). Analogues of ST have been generated by cloning and by chemical techniques. Small peptide fragments of the native ST structure which include the structural determinant that confers binding activity may be constructed. Once a structure is identified which binds to ST receptors, non-peptide analogues mimicking that structure in space are designed.

U.S. Pat. Nos. 5,140,102 and 7,041,786, and U.S. Published Applications US 2004/0258687 A1 and US 2005/0287067 A1 also refer to compounds which may bind to and activate guanylyl cyclase C.

SEQ ID NO:1 discloses a nucleotide sequence which encodes 19 amino acid ST, designated ST Ia, reported by So and McCarthy (1980) Proc. Natl. Acad. Sci. USA 77:4011, which is incorporated herein by reference.

The amino acid sequence of ST Ia is disclosed in SEQ ID NO:2.

SEQ ID NO:3 discloses the amino acid sequence of an 18 amino acid peptide which exhibits ST activity, designated ST I*, reported by Chan and Giannella (1981) J. Biol. Chem. 256:7744, which is incorporated herein by reference.

SEQ ID NO:4 discloses a nucleotide sequence which encodes 19 amino acid ST, designated ST Ib, reported by Mosely et al. (1983) Infect. Immun. 39:1167, which is incorporated herein by reference.

The amino acid sequence of ST Ib is disclosed in SEQ ID NO:5.

A 15 amino acid peptide called guanylin which has about 50% sequence homology to ST has been identified in mammalian intestine (Currie, M. G. et al. (1992) Proc. Natl. Acad. Sci. USA 89:947-951, which is incorporated herein by reference). Guanylin binds to ST receptors and activates guanylate cyclase at a level of about 10- to 100-fold less than native ST. Guanylin may not exist as a 15 amino acid peptide in the intestine but rather as part of a larger protein in that organ. The amino acid sequence of guanylin from rodent is disclosed as SEQ ID NO:6.

SEQ ID NO:7 is an 18 amino acid fragment of SEQ ID NO:2. SEQ ID NO:8 is a 17 amino acid fragment of SEQ ID NO:2. SEQ ID NO:9 is a 16 amino acid fragment of SEQ ID NO:2. SEQ ID NO:10 is a 15 amino acid fragment of SEQ ID NO:2. SEQ ID NO:11 is a 14 amino acid fragment of SEQ ID NO:2. SEQ ID NO:12 is a 13 amino acid fragment of SEQ ID NO:2. SEQ ID NO:13 is an 18 amino acid fragment of SEQ ID NO:2. SEQ ID NO:14 is a 17 amino acid fragment of SEQ ID NO:2. SEQ ID NO:15 is a 16 amino acid fragment of SEQ ID NO:2. SEQ ID NO:16 is a 15 amino acid fragment of SEQ ID NO:2. SEQ ID NO:17 is a 14 amino acid fragment of SEQ ID NO:2.

SEQ ID NO:18 is a 17 amino acid fragment of SEQ ID NO:3. SEQ ID NO:19 is a 16 amino acid fragment of SEQ ID NO:3. SEQ ID NO:20 is a 15 amino acid fragment of SEQ ID NO:3. SEQ ID NO:21 is a 14 amino acid fragment of SEQ ID NO:3. SEQ ID NO:22 is a 13 amino acid fragment of SEQ ID NO:3. SEQ ID NO:23 is a 17 amino acid fragment of SEQ ID NO:3. SEQ ID NO:24 is a 16 amino acid fragment of SEQ ID NO:3. SEQ ID NO:25 is a 15 amino acid fragment of SEQ ID NO:3. SEQ ID NO:26 is a 14 amino acid fragment of SEQ ID NO:3.

SEQ ID NO:27 is an 18 amino acid fragment of SEQ ID NO:5. SEQ ID NO:28 is a 17 amino acid fragment of SEQ ID NO:5. SEQ ID NO:29 is a 16 amino acid fragment of SEQ ID NO:5. SEQ ID NO:30 is a 15 amino acid fragment of SEQ ID NO:5. SEQ ID NO:31 is a 14 amino acid fragment of SEQ ID NO:5. SEQ ID NO:32 is a 13 amino acid fragment of SEQ ID NO:5. SEQ ID NO:33 is an 18 amino acid fragment of SEQ ID NO:5. SEQ ID NO:34 is a 17 amino acid fragment of SEQ ID NO:5. SEQ ID NO:35 is a 16 amino acid fragment of SEQ ID NO:5. SEQ ID NO:36 is a 15 amino acid fragment of SEQ ID NO:5. SEQ ID NO:37 is a 14 amino acid fragment of SEQ ID NO:5.

SEQ ID NO:27, SEQ ID NO:31, SEQ ID NO:36 AND SEQ ID NO:37 are disclosed in Yoshimura, S., et al. (1985) FEBS Lett. 181:138, which is incorporated herein by reference.

SEQ ID NO:38, SEQ ID NO:39 and SEQ ID NO:40, which are derivatives of SEQ ID NO:3, are disclosed in Waldman, S. A. and O'Hanley, P. (1989) Infect. Immun. 57:2420, which is incorporated herein by reference.

SEQ ID NO:41, SEQ ID NO:42, SEQ ID NO:43, SEQ ID NO:44 and SEQ ID NO:45, which are a derivatives of SEQ ID NO:3, are disclosed in Yoshimura, S., et al. (1985) FEBS Lett. 181:138, which is incorporated herein by reference.

SEQ ID NO:46 is a 25 amino acid peptide derived from Y. enterocolitica which binds to the ST receptor.

SEQ ID NO:47 is a 16 amino acid peptide derived from V. cholerae which binds to the ST receptor. SEQ ID NO:47 is reported in Shimonishi, Y., et al. FEBS Lett. 215:165, which is incorporated herein by reference.

SEQ ID NO:48 is an 18 amino acid peptide derived from Y. enterocolitica which binds to the ST receptor. SEQ ID NO:48 is reported in Okamoto, K., et al. Infec. Immun. 55:2121, which is incorporated herein by reference.

SEQ ID NO:49, is a derivative of SEQ ID NO:5. SEQ ID NO:50, SEQ ID NO:51, SEQ ID NO:52 and SEQ ID NO:53 are derivatives. SEQ ID NO:54 is the amino acid sequence of guanylin from human.

A 15 amino acid peptide called uroguanylin has been identified in mammalian intestine from opossum (Hamra, S. K. et al. (1993) Proc. Natl. Acad. Sci. USA 90:10464-10468, which is incorporated herein by reference; see also Forte L. and M. Curry 1995 FASEB 9:643-650; which is incorporated herein by reference). SEQ ID NO:55 is the amino acid sequence of uroguanylin from opossum.

A 16 amino acid peptide called uroguanylin has been identified in mammalian intestine from human (Kita, T. et al. (1994) Amer. J. Physiol. 266:F342-348, which is incorporated herein by reference; see also Forte L. and M. Curry 1995 FASEEB 9:643-650; which is incorporated herein by reference). SEQ ID NO:56 is the amino acid sequence of uroguanylin from human.

SEQ ID NO:57 is the amino acid sequence of proguanylin, a guanylin precursor which is processed into active guanylin.

SEQ ID NO:58 is the amino acid sequence of prouroguanylin, a uroguanylin precursor which is processed into active uroguanylin.

Although proguanylin and prouroguanylin are precursors for mature guanylin and mature uroguanylin respectively, they may be used as GCC agonists as described herein provide they are delivered such that they can be processed into the mature peptides.

U.S. Pat. Nos. 5,140,102, 7,041,786 and 7,304,036, and U.S. Published Applications US 2004/0258687, US 2005/0287067, 20070010450, 20040266989, 20060281682, 20060258593, 20060094658, 20080025966, 20030073628, 20040121961 and 20040152868, which are each incorporated herein by reference, also refer to compounds which may bind to and activate guanylyl cyclase C.

In addition to human guanylin and human uroguanylin, guanylin or uroguanylin may be isolated or otherwise derived from other species such as cow, pig, goat, sheep, horse, rabbit, bison, etc. Such guanylin or uroguanylin may be administered to individuals including humans.

Antibodies including GCC binding antibody fragments can also be GCC agonists. Antibodies may include for example polyclonal and monoclonal antibodies including chimeric, primatized, humanized or human monoclonal antibodies as well as antibody fragments that bind to GCC with agonist activity such as CDRs, FAbs, F(Ab), Fv's including single chain Fv and the like. Antibodies may be IgE, IgA or IgM for example.

To reduce side effects caused by intestinal cell death, GCC agonists are delivered to the colorectal track by the oral delivery of such GCC agonists. ST peptides and the endogenous GCC agonist peptides, for example, are stable and can survive the stomach acid and pass through the small intestine to the colorectal track. Sufficient dosages are provided to ensure that GCC agonist reaches the large intestine in sufficient quantities to induce accumulation of cGMP in those cells as well.

GCC agonists such as for example ST, guanylin and uroguanylin, can survive the gastric environment. Thus, they may be administered without coating or protection against stomach acid. However, in order to more precisely control the release of GCC agonists administered orally, the GCC agonist may be enterically coated so that some or all of the GCC agonist is released after passing through the stomach. Such enteric coating may also be designed to provide a sustained or extended release of the GCC agonist over the period of time with which the coated GCC agonist passes through the intestines. In some embodiments, the GCC agonist may be formulated to ensure release of some compound upon entering the large intestine. In some embodiments, the GCC agonist may be delivered rectally.

Most enteric coatings are intended to protect contents from stomach acid. Accordingly, they are designed to release active agent upon passing through the stomach. The coatings and encapsulations used herein are provided to begin releasing the GCC agonist in the small intestine and preferably over an extended period of time so that GCC agonist concentrations can be maintained t an effective level for a greater period of time.

According to some embodiments, the GCC agonists are coated or encapsulated with a sufficient amount of coating material that the time required for the coating material to dissolve and release the GCC agonists corresponds with the time required for the coated or encapsulated composition to travel from the mouth to intestines.

According to some embodiments, the GCC agonists are coated or encapsulated with coating material that does not fully dissolve and release the GCC agonists until it comes in contact with conditions present in the small intestine. Such conditions may include the presence of enzymes in the colorectal track, pH, tonicity, or other conditions that vary relative to the stomach.

According to some embodiments, the GCC agonists are coated or encapsulated with coating material that is designed to dissolve in stages as it passes from stomach to small intestine to large intestine.

According to some embodiments, the GCC agonists are complexed with another molecular entity such that they are inactive until the GCC agonists cease to be complexed with molecular entity and are present in active form. In such embodiments, the GCC agonists are administered as “prodrugs” which become processed into active GCC agonists in the colorectal track.

Examples of technologies which may be used to formulate GCC agonists for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

Examples of technologies which may be used to formulate GCC agonists or inducers for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. No. 6,309,666; U.S. Pat. No. 6,569,463, U.S. Pat. No. 6,214,378; U.S. Pat. No. 6,248,363; U.S. Pat. No. 6,458,383, U.S. Pat. No. 6,531,152, U.S. Pat. No. 5,576,020, U.S. Pat. No. 5,654,004, U.S. Pat. No. 5,294,448, U.S. Pat. No. 6,309,663, U.S. Pat. No. 5,525,634, U.S. Pat. No. 6,248,362, U.S. Pat. No. 5,843,479, and U.S. Pat. No. 5,614,220, which are each incorporated herein by reference.

In some embodiments, the effective amount is delivered so that sufficient accumulation of cGMP results, for at least a period of 2 hours. In some embodiments, the effective amount is present for up to 12 hours to several days. Multiple doses may be administered to maintain levels such that the amount of GCC agonist present, either free or bound to GCC, remains ay or above the effective dose. In some embodiments, an initial loading dose and/or multiple administrations are required for cells of the intestine to become protected from radiation and chemotherapy induced cell death. After cells exposed to GCC agonist become resistant to cell death induced by radiation and chemotherapy, radiation or chemotherapeutics may be administered, in some cases in doses much higher than could be tolerated by patients who have not been pretreated with GCC agonist.

In some embodiments, GCC agonists which are peptides may be administered in an amount ranging from 100 ug to 1 gram every 4-48 hours. In some embodiments, GCC agonists are administered in an amount ranging from 1 mg to 750 mg every 4-48 hours. In some embodiments, GCC agonists are administered in an amount ranging from 10 mg to 500 mg every 4-48 hours. In some embodiments, GCC agonists are administered in an amount ranging from 50 mg to 250 mg every 4-48 hours. In some embodiments, GCC agonists are administered in an amount ranging from 75 mg to 150 mg every 4-48 hours.

In some embodiments, doses are administered every 4 or more hours. In some embodiments, doses are administered every 6 or more hours. In some embodiments, doses are administered every 8 or more hours. In some embodiments, doses are administered every 12 or more hours. In some embodiments, doses are administered every 24 or more hours. In some embodiments, doses are administered every 48 or more hours. In some embodiments, doses are administered every 4 hours or less. In some embodiments, doses are administered every 6 hours or less. In some embodiments, doses are administered every 8 hours or less. In some embodiments, doses are administered every 12 hours or less. In some embodiments, doses are administered every 24 hours or less. In some embodiments, doses are administered every 48 hours or less.

In some embodiments, additives or co-agents are administered in combination with GCC agonists to a minimize diarrhea or cramping/intestinal contractions-increased motility. For example, the individual may be administered a compound that before, simultaneously or after administration with a compound that relieves diarrhea. Such anti-diarrheal component may be incorporated in the formulation. Anti-diarrheal compounds and preparations, such as loperamide, bismuth subsalicylate and probiotic treatments such as strains of Lactobaccilus, are well known and widely available.

According to some aspects of the invention, innocuous bacteria of species that normally populate the colon are provided with genetic information needed to produce a guanylyl cyclase C agonist in the colon, making such guanylyl cyclase C agonist available to produce the effect of activating the guanylyl cyclase C on colon cells. The existence of a population of bacteria which can produce guanylyl cyclase C agonist provides a continuous administration of the guanylyl cyclase C agonist. In some embodiments, the nucleic acid sequences that encode the guanylyl cyclase C agonist may be under the control of an inducible promoter. Accordingly, the individual may turn expression on or off depending upon whether or not the inducer is ingested. In some embodiments, the inducer is formulated to be specifically released in the colon, thereby preventing induction of expression by the bacteria that may be populating other sites such as the small intestine. In some embodiments, the bacteria are is sensitive to a particular drug or auxotrophic such that it can be eliminated by administration of the drug or withholding an essential supplement.

The technology for introducing expressible forms of genes into bacteria is well known and the materials needed are widely available.

In some embodiments, bacteria which comprise coding sequences for a GCC agonist may be those of a species which commonly inhabits the intestinal track of an individual. Common gut flora include species from the genera Bacteroides, Clostridium, Fusobacterium, Eubacterium, Ruminococcus, Peptococcus, Peptostreptococcus, Bifidobacteriu, Escherichia and Lactobacillus. In some embodiments, the bacteria is selected from a strain known to be useful as a probiotic. Examples of species of bacteria used as compositions for administration to humans include Bifidobacterium bifidum; Escherichia coli, Lactobacillus acidophilus, Lactobacillus rhamnosus, Lactobacillus casei, and Lactobacillus johnsonii. Other species include Lactobacillus bulgaricus, Streptococcus thermophilus, Bacillus coagulans and Lactobacillus bifidus. Examples of strains of bacteria used as compositions for administration to humans include: B. infantis 35624, (Align); Lactobacillus plantarum 299V; Bifidobacterium animalis DN-173 010; Bifidobacterium animalis DN 173 010 (Activia Danone); Bifidobacterium animalis subsp. lactis BB-12 (Chr.Hansen); Bifidobacterium breve Yakult Bifiene Yakult; Bifidobacterium infantis 35624 Bifidobacterium lactis HN019 (DR10) Howaru™ Bifido Danisco; Bifidobacterium longum BB536; Escherichia coli Nissle 1917; Lactobacillus acidophilus LA-5 Chr. Hansen; Lactobacillus acidophilus NCFM Rhodia Inc.; Lactobacillus casei DN114-001; Lactobacillus casei CRL431 Chr. Hansen; Lactobacillus casei F19 Cultura Arla Foods; Lactobacillus casei Shirota Yakult Yakult; Lactobacillus casei immunitass Actimel Danone; Lactobacillus johnsonnii Lal (=Lactobacillus LC1) Nestlé; Lactobacillus plantarum 299V ProViva Probi IBS; Lactobacillus reuteri ATTC 55730 BioGaia Biologics; Lactobacillus reuteri SD2112; Lactobacillus rhamnosus ATCC 53013 Vifit and others Valio; Lactobacillus rhamnosus LB21 Verum Norrmejerier; Lactobacillus salivarius UCC118; Lactococcus lactis L1A Verum Norrmejerier; Saccharomyces cerevisiae (boulardii) lyo; Streptococcus salivarius ssp thermophilus; Lactobacillus rhamnosus GR-1; Lactobacillus reuteri RC-14; Lactobacillus acidophilus CUL60; Bifidobacterium bifidum CUL 20; Lactobacillus helveticus R0052; and Lactobacillus rhamnosus R0011.

The following U.S. patents, which are each incorporated herein by reference, disclose non-pathogenic bacteria which can be administered to individuals. U.S. Pat. No. 6,200,609; U.S. Pat. No. 6,524,574, U.S. Pat. No. 6,841,149, U.S. Pat. No. 6,878,373, U.S. Pat. No. 7,018,629, U.S. Pat. No. 7,101,565, U.S. Pat. No. 7,122,370, U.S. Pat. No. 7,172,777, U.S. Pat. No. 7,186,545, U.S. Pat. No. 7,192,581, U.S. Pat. No. 7,195,906, U.S. Pat. No. 7,229,818, and U.S. Pat. No. 7,244,424.

Accordingly the aspects of the invention, bacteria would first be provided with genetic material encoding a GCC agonist in a form that would permit expression le of the agonist peptide within the bacteria, either constitutively or upon induction by the presence of an inducer that would turn on an inducible promoter.

Some embodiments comprise inducible regulatory elements such as inducible promoters. Typically, an inducible promoter is one in which an agent, when present, interacts with the promoter such that expression of the coding sequence operably linked to the promoter proceeds. Alternatively, an inducible promoter can include a repressor which is an agent that interacts with the promoter and prevent expression of the coding sequence operably linked to the promoter. Removal of the repressor results in expression of the coding sequence operably linked to the promoter.

The agents that induce an inducible promoter are preferably not naturally present in the organism where expression of the transgene is sought. Accordingly, the transgene is only expressed when the organism is affirmatively exposed to the inducing agent. Thus, in a bacterium that includes a transgene operably linked to an inducible promoter, when the bacterium is living within the gut of an individual, the promoter may be turned on and the transgene expressed when the individual ingests the inducing agent.

The agents that induce an inducible promoter are preferably not toxic. Thus, in a bacterium that includes a transgene operably linked to an inducible promoter, the inducing agent is preferably not toxic to the individual in whose gut the bacterium is living such that when the individual ingests the inducing agent to turn on expression of the transgene the inducing agent dose not have any severe toxic side effects on the individual.

The agents that induce an inducible promoter preferably affect only the expression of the gene of interest. Thus, in a bacterium that includes a transgene operably linked to an inducible promoter, the inducing agent does not have any significant affect on the expression of any other genes in the individual.

The agents that induce an inducible promoter preferably are easy to apply or removal. Thus, in a bacterium that includes a transgene operably linked to an inducible promoter that is living in the gut of an individual, the inducing agent is preferably an agent that can be easily delivered to the gut and that can be removed, either by affirmative neutralization for example or by metabolism/passing such that gene expression can be controlled.

The agents that induce an inducible promoter preferably induce a clearly detectable expression pattern of either high or very low gene expression.

In some preferred embodiments, the chemically-regulated promoters are derived from organisms distant in evolution to the organisms where its action is required. Examples of inducible or chemically-regulated promoters include tetracycline-regulated promoters. Tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. The Tetracycline resistance operon is carried by the Escherichia coli transposon (Tn) 10. This operon has a negative mode of operation. The interaction between a repressor protein encoded by the operon, TetR, and a DNA sequence to which it binds, the tet operator (tetO), represses the activity of a promoter placed near the operator. In the absence of an inducer, TetR binds to tetO and prevents transcription. Transcription can be turned on when an inducer, such as tetracycline, binds to TetR and causes a conformation change that prevents TetR from remaining bound to the operator. When the operator site is not bound, the activity of the promoter is restored. Tetracycline, the antibiotic, has been used to create two beneficial enhancements to inducible promoters. One enhancement is an inducible on or off promoter. The investigators can choose to have the promoter always activated until Tet is added or always inactivated until Tet is added. This is the Tet on/off promoter. The second enhancement is the ability to regulate the strength of the promoter. The more Tet added, the stronger the effect.

Examples of inducible or chemically-regulated promoters include Steroid-regulated promoters. Steroid-responsive promoters are provided for the modulation of gene expression include promoters based on the rat glucocorticoid receptor (GR); human estrogen receptor (ER); ecdysone receptors derived from different moth species; and promoters from the steroid/retinoid/thyroid receptor superfamily. The hormone binding domain (HBD) of GR and other steroid receptors can also be used to regulate heterologous proteins in cis, that is, operatively linked to protein-encoding sequences upon which it acts. Thus, the HBD of GR, estrogen receptor (ER) and an insect ecdysone receptor have shown relatively tight control and high inducibility.

Examples of inducible or chemically-regulated promoters include metal-regulated promoters. Promoters derived from metallothionein (proteins that bind and sequester metal ions) genes from yeast, mouse and human are examples of promoters in which the presence of metals induces gene expression.

IPTG is a classic example of a compound added to cells to activate a promoter. IPTG can be added to the cells to activate the downstream gene or removed to inactivate the gene.

U.S. Pat. No. 6,180,391, which is incorporated herein by reference, refers to the a copper-inducible promoter.

U.S. Pat. No. 6,943,028, which is incorporated herein by reference, refers to highly efficient controlled expression of exogenous genes in E. coli.

U.S. Pat. No. 6,180,367, which is incorporated herein by reference, refers to a process for bacterial production of polypeptides.

Other examples of inducible promoters suitable for use with bacterial hosts include the beta.-lactamase and lactose promoter systems (Chang et al., Nature, 275: 615 (1978, which is incorporated herein by reference); Goeddel et al., Nature, 281: 544 (1979), which is incorporated herein by reference), the arabinose promoter system, including the araBAD promoter (Guzman et al., J. Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the P.sub.LtetO-1 and P.sub.lac/are-1 promoters (Lutz and Bujard, Nucleic Acids Res., 25: 1203-1210 (1997), which is incorporated herein by reference), and hybrid promoters such as the tac promoter. deBoer et al., Proc. Natl. Acad. Sci. USA, 80: 21-25 (1983), which is incorporated herein by reference. However, other known bacterial inducible promoters and low-basal-expression promoters are suitable.

U.S. Pat. No. 6,083,715, which is incorporated herein by reference, refers to methods for producing heterologous disulfide bond-containing polypeptides in bacterial cells.

U.S. Pat. No. 5,830,720, which is incorporated herein by reference, refers to recombinant DNA and expression vector for the repressible and inducible expression of foreign genes.

U.S. Pat. No. 5,789,199, which is incorporated herein by reference, refers to a process for bacterial production of polypeptides.

U.S. Pat. No. 5,085,588, which is incorporated herein by reference, refers to bacterial promoters inducible by plant extracts.

U.S. Pat. No. 6,242,194, which is incorporated herein by reference, refers to probiotic bacteria host cells that contain a DNA of interest operably associated with a promoter of the invention can be orally administered to a subject.

U.S. Pat. No. 5,364,780, which is incorporated herein by reference, refers to external regulation of gene expression by inducible promoters.

U.S. Pat. No. 5,639,635, which is incorporated herein by reference, refers to a process for bacterial production of polypeptides.

U.S. Pat. No. 5,789,199, which is incorporated herein by reference, refers to a process for bacterial production of polypeptides.

U.S. Pat. No. 5,689,044, which is incorporated herein by reference, refers to chemically inducible promoter of a plant PR-1 gene.

U.S. Pat. No. 5,063,154, which is incorporated herein by reference, refers to a pheromone-inducible yeast promoter.

U.S. Pat. No. 5,658,565, which is incorporated herein by reference, refers to an inducible nitric oxide synthase gene.

U.S. Pat. Nos. 5,589,392, 6,002,069, 5,693,531, 5,480,794, 6,171,816, 6,541,224, 6,495,318, 5,498,538, 5,747,281, 6,635,482 and 5,364,780, which are each incorporated herein by reference, each refer to an IPTG-inducible promoters.

U.S. Pat. Nos. 6,420,170, 5,654,168, 5,912,411, 5,891,718, 6,133,027, 5,739,018, 6,136,954, 6,258,595, 6,002,069 and 6,025,543, which are each incorporated herein by reference, each refer to an tetracycline-inducible promoters.

### Guanylyl Cyclase a (GCA) Agonists (ANP, BNP)

Guanylyl cyclase-A/natriuretic peptide receptor-A (GCA) is a cellular protein involved in maintaining renal and cardiovascular homeostasis. GCA is a receptor found in kidney cells that binds to and is activated by two peptide made in the heart. Atrial natriuretic peptide (ANP, also referred to as cardiac atrial natriuretic peptide) is stored in the heart as pro-ANP and when released, is processed into mature ANP. B-type natriuretic peptide (BNP, also referred to as brain natriuretic peptide) is also produced in the heart. When ANP or BNP bounds to GCA, the GCA-expressing cells produce cGMP as a second messenger. Thus, ANP and BNP are GCA agonists which activate GCA and lead to accumulation of cGMP in cells expressing GCA.

ANP analogs that are GCA agonists are disclosed in Schiller P W, et al. Superactive analogs of the atrial natriuretic peptide (ANP), Biochem Biophys Res Commun. 1987 Mar. 13; 143(2):499-505; Schiller P W, et al. Synthesis and activity profiles of atrial natriuretic peptide (ANP) analogs with reduced ring size. Biochem Biophys Res Commun. 1986 Jul. 31; 138(2):880-6; Goghari M H, et al. Synthesis and biological activity profiles of atrial natriuretic factor (ANF) analogs., Int J Pept Protein Res. 1990 August; 36(2):156-60; Bovy P R, et al. A synthetic linear decapeptide binds to the atrial natriuretic peptide receptors and demonstrates cyclase activation and vasorelaxant activity. J Biol. Chem. 1989 Dec. 5; 264(34):20309-13, and Schoenfeld et al. Molecular Pharmacology January 1995 vol. 47 no. 1 172-180.

### Guanylyl Cyclase B (GCB) Agonists (CNP)

Guanylyl cyclase B (GCB) is also referred to as natriuretic peptide receptor B, atrionatriuretic peptide receptor B and NPR2. GCB is the receptor for a small peptide (C-type natriuretic peptide) produced locally in many different tissues. GCA expression is reported in the kidney, ovarian cells, aorta, chondrocytes, the corpus cavernosum, the pineal gland among other.

While GCB is reported to bind to and be activated by ANP and BNP, C-type natriuretic peptide (CNP) is the most potent activator of GCB. ANP, BNP and CNP are GCB agonists. U.S. Pat. No. 5,434,133 and Furuya, M et al. Biochemical and Biophysical Research Communications, Volume 183, Issue 3, 31 Mar. 1992, Pages 964-969, disclose CNP analogs.

### Soluble Guanylyl Cyclase Activators (Nitric Oxide, Nitrovasodilators, Protoprophyrin IX, and Direct Activators)

Soluble guanylyl cyclase (sGC) is heterodimeric protein made up of an alpha domain with C terminal region that has cyclase activity and a heme-binding beta domain which also has with a C terminal region that has cyclase activity. The sGC which is the only known receptor for nitric oxide has one heme per dimmer. The heme moiety in Fe(II) form is the target of NO. NO binding results in activation of sGC, i.e. a substantial increase in sGC activity. Activation of sGC is involved in vasodilation.

YC-1, which is 5-[1-(phenylmethyl)-1H-indazol-3-yl]-2-furanmethanol, is an nitric oxide (NO)-independent activator of soluble guanylyl cyclase. Ko F N et al. YC-1, a novel activator of platelet guanylate cyclase. Blood. 1994 Dec. 15; 84(12):4226-33.

Two drugs that activate sGC are cinaciguat (4-({(4-carboxybutyl)[2-(2-{[4-(2-phenylethyl) phenyl]methoxy}phenyl)ethyl]amino}methyl)benzoic acid) WO-0119780, U.S. Pat. Nos. 7,087,644, 7,517,896 WO 20008003414 WO 2008148474 and riociguat, (Methyl N-[4,6-Diamino-2-[1-[(2-fluorophenyl)methyl]-1H-pyrazolo[3,4-b]pyridin-3-yl]-5-pyrimidinyl]-N-methyl-carbaminate) WO-03095451, which has been granted in the US as U.S. Ser. No. 07/173,037.

Other examples of sGC activators include 3-(5′-hydroxymethyl-2′-furyl)-1-benzylindazole (YC-1, Wu et al., Blood 84 (1994), 4226; Mulsch et al., Brit. J. Pharmacol. 120 (1997), 681), fatty acids (Goldberg et al, J. Biol. Chem. 252 (1977), 1279), diphenyliodonium hexafluorophosphate (Pettibone et al., Eur. J. Pharmacol. 116 (1985), 307), isoliquiritigenin (Yu et. al., Brit. J. Pharmacol. 114 (1995), 1587) and various substituted pyrazole derivatives (WO 98/16223). In addition, WO 98/16507, WO 98/23619, WO 00/06567, WO 00/06568, WO 00/06569, WO 00/21954 WO 02/42299, WO 02/42300, WO 02/42301, WO 02/42302, WO 02/092596 and WO 03/004503 describe pyrazolopyridine derivatives as stimulators of soluble guanylate cyclase. Also described inter alia therein are pyrazolopyridines having a pyrimidine residue in position 3. Compounds of this type have very high in vitro activity in relation to stimulating soluble guanylate cyclase. However, it has emerged that these compounds have disadvantages in respect of their in vivo properties such as, for example, their behavior in the liver, their pharmacokinetic behavior, their dose-response relation or their metabolic pathway.

Other sGC activators are disclosed in 0. V. Evgenov et al., Nature Rev. Drug Disc. 5 (2006), 755; and US Published Patent Application Publication Nos. 20110034450, 20100210643, 20100197680, 20100168240, 20100144864, 20100144675, 20090291993, 20090286882, 20090215843, 20080

### PDE Inhibitors

In some embodiments, the active agent comprises PDE inhibitors including, for example, nonselective phosphodiesterase inhibitors, PDE1 selective inhibitors, PDE2 selective inhibitors, PDE3 selective inhibitors, PDE4 selective inhibitors, PDE5 selective inhibitors, and PDE10 selective inhibitors.

PDE inhibitors are generally discussed in the following references which are each incorporated herein by reference: Uzunov, P. and Weiss, B.: Separation of multiple molecular forms of cyclic adenosine 3′,5′-monophosphate phosphodiesterase in rat cerebellum by polyacrylamide gel electrophoresis. Biochim. Biophys. Acta 284:220-226, 1972; Weiss, B.: Differential activation and inhibition of the multiple forms of cyclic nucleotide phosphodiesterase. Adv. Cycl. Nucl. Res. 5:195-211, 1975; Fertel, R. and Weiss, B.: Properties and drug responsiveness of cyclic nucleotide phosphodiesterases of rat lung. Mol. Pharmacol. 12:678-687, 1976; Weiss, B. and Hait, W. N.: Selective cyclic nucleotide phosphodiesterase inhibitors as potential therapeutic agents. Ann. Rev. Pharmacol. Toxicol. 17:441-477, 1977; Essayan D M. (2001). “Cyclic nucleotide phosphodiesterases.”. J Allergy Clin Immunol. 108 (5): 671-80; Deree J, Martins J O, Melbostad H, Loomis W H, Coimbra R. (2008). “Insights into the Regulation of TNF-α Production in Human Mononuclear Cells: The Effects of Non-Specific Phosphodiesterase Inhibition”. Clinics (Sao Paulo). 63 (3): 321-8; Marques L J, Zheng L, Poulakis N, Guzman J, Costabel U (February 1999). “Pentoxifylline inhibits TNF-alpha production from human alveolar macrophages”. Am. J. Respir. Crit. Care Med. 159 (2): 508-11; Peters-Golden M, Canetti C, Mancuso P, Coffey M J. (2005). “Leukotrienes: underappreciated mediators of innate immune responses”. J Immunol. 174 (2): 589-94; Daly J W, Jacobson K A, Ukena D. (1987). “Adenosine receptors: development of selective agonists and antagonists”. Prog Clin Biol Res. 230 (1): 41-63; MacCorquodale D W. THE SYNTHESIS OF SOME ALKYLXANTHINES. Journal of the American Chemical Society. 1929 July; 51(7):2245-2251; WO/1985/002540; U.S. Pat. No. 4,288,433; Daly J W, Padgett W L, Shamim M T (July 1986). “Analogues of caffeine and theophylline: effect of structural alterations on affinity at adenosine receptors”. Journal of Medicinal Chemistry 29 (7): 1305-8; Daly J W, Jacobson K A, Ukena D (1987). “Adenosine receptors: development of selective agonists and antagonists”. Progress in Clinical and Biological Research 230: 41-63; Choi O H, Shamim M T, Padgett W L, Daly J W (1988). “Caffeine and theophylline analogues: correlation of behavioral effects with activity as adenosine receptor antagonists and as phosphodiesterase inhibitors”. Life Sciences 43 (5): 387-98; Shamim M T, Ukena D, Padgett W L, Daly J W (June 1989). “Effects of 8-phenyl and 8-cycloalkyl substituents on the activity of mono-, di-, and trisubstituted alkylxanthines with substitution at the 1-, 3-, and 7-positions”. Journal of Medicinal Chemistry 32 (6): 1231-7; Daly J W, Hide I, Müller C E, Shamim M (1991). “Caffeine analogs: structure-activity relationships at adenosine receptors”. Pharmacology 42 (6): 309-21; Ukena D, Schudt C, Sybrecht G W (February 1993). “Adenosine receptor-blocking xanthines as inhibitors of phosphodiesterase isozymes”. Biochemical Pharmacology 45 (4): 847-51. doi:10.1016/0006-2952(93)90168-V; Daly J W (July 2000). “Alkylxanthines as research tools”. Journal of the Autonomic Nervous System 81 (1-3): 44-52. doi:10.1016/50165-1838(00)00110-7; Daly J W (August 2007). “Caffeine analogs: biomedical impact”. Cellular and Molecular Life Sciences: CMLS 64 (16): 2153-69; González M P, Terán C, Teijeira M (May 2008). “Search for new antagonist ligands for adenosine receptors from QSAR point of view. How close are we?”. Medicinal Research Reviews 28 (3): 329-71; Baraldi P G, Tabrizi M A, Gessi S, Borea P A (January 2008). “Adenosine receptor antagonists: translating medicinal chemistry and pharmacology into clinical utility”. Chemical Reviews 108 (1): 238-63; de Visser Y P, Walther F J, Laghmani E H, van Wijngaarden S, Nieuwland K, Wagenaar G T. (2008). “Phosphodiesterase-4 inhibition attenuates pulmonary inflammation in neonatal lung injury”. Eur Respir J 31 (3): 633-644; Yu M C, Chen J H, Lai C Y, Han C Y, Ko W C. (2009). “Luteolin, a non-selective competitive inhibitor of phosphodiesterases 1-5, displaced [(3)H]-rolipram from high-affinity rolipram binding sites and reversed xylazine/ketamine-induced anesthesia”. Eur J. Pharmacol. 627 (1-3): 269-75; Bobon D, Breulet M, Gerard-Vandenhove M A, Guiot-Goffioul F, Plomteux G, Sastre-y-Hernandez M, Schratzer M, Troisfontaines B, von Frenckell R, Wachtel H. (1988). “Is phosphodiesterase inhibition a new mechanism of antidepressant action? A double blind double-dummy study between rolipram and desipramine in hospitalized major and/or endogenous depressives”. Eur Arch Psychiatry Neurol Sci. 238 (1): 2-6; Maxwell C R, Kanes S J, Abel T, Siegel S J. (2004). “Phosphodiesterase inhibitors: a novel mechanism for receptor-independent antipsychotic medications”. Neuroscience. 129 (1): 101-7; Kanes S J, Tokarczyk J, Siegel S J, Bilker W, Abel T, Kelly M P. (2006). “Rolipram: A specific phosphodiesterase 4 inhibitor with potential antipsychotic activity”. Neuroscience. 144 (1): 239-46; and Vecsey C G, Baillie G S, Jaganath D, Havekes R, Daniels A, Wimmer M, Huang T, Brown K M, Li X Y, Descalzi G, Kim S S, Chen T, Shang Y Z, Zhuo M, Houslay M D, Abel T. (2009). “Sleep deprivation impairs cAMP signaling in the hippocampus”. Nature. 461 (7267): 1122-1125.

In addition to activation of guanylyl cyclases, cGMP levels can be elevated and cells protected from chemotherapeutics and radiation therapy using PDE such as PDE 1, PDE2, PDE3, PDE4, PDE5 and PDE10 inhibitors. The breakdown of cGMP is controlled by a family of phosphodiesterase (PDE) isoenzymes. To date, seven members of the family have been described (PDE I-VII) the distribution of which varies from tissue to tissue (Beavo & Reifsnyder (1990) TIPS, 11:150-155 and Nicholson et al (1991) TIPS, 12: 19-27). Specific inhibitors of PDE isoenzymes may be useful to achieve differential elevation of cGMP in different tissues. Some PDE inhibitors specifically inhibit breakdown of cGMP while not effecting cAMP. In some embodiments, possible PDE inhibitors may be PDE3 inhibitors, PDE4 inhibitors, PDE5 inhibitors, PDE3/4 inhibitors or PDE3/4/5 inhibitors.

PDE inhibitors which elevate cGMP specifically are disclosed in U.S. Pat. Nos. 6,576,644, 7,384,958, 7,276,504, 7,273,868, 7,220,736, 7,098,209, 7,087,597, 7,060,721, 6,984,641, 6,930,108, 6,911,469, 6,784,179, 6,656,945, 6,642,244, 6,476,021, 6,326,379, 6,316,438, 6,306,870, 6,300,335, 6,218,392, 6,197,768, 6,037,119, 6,025,494, 6,018,046, 5,869,516, 5,869,486, 5,716,993. Other examples include compounds disclosed in WO 96/05176 and U.S. Pat. No. 6,087,368, U.S. Pat. Nos. 4,101,548, 4,001,238, 4,001,237, 3,920,636, 4,060,615, 4,209,623, 5,354,571, 3,031,450, 3,322,755, 5,401,774, 5,147,875, 4,885,301, 4,162,316, 4,047,404, 5,614,530, 5,488,055, 4,880,810, 5,439,895, 5,614,627, GB 2 063 249, EP 0 607 439, WO 97/03985, EP 0 395 328, EP 0 428 268, PCT WO 93/12095, WO 93/07149, EP 0 349 239, EP 0 352 960, EP 0 526 004, EP 0 463 756, EP 0 607 439, WO 94/05661, EP 0 351 058, EP 0 347 146, WO 97/03985, WO 97/03675, WO 95/19978, WO 98/08848, WO 98/16521, EP 0 722 943, EP 0 722 937, EP 0 722 944, WO 98/17668, WO 97/24334, WO 98/06722, PCT/JP97/03592, WO 98/23597, WO 94/29277, WO 98/14448, WO 97/03070, WO 98/38168, WO 96/32379, and PCT/GB98/03712. PDE inhibitors may include those disclosed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 and WO9307124, EP0163965, EP0393500, EP0510562, EP0553174, WO9501338 and WO9603399.

Examples of nonselective phosphodiesterase inhibitors include: methylated xanthines and derivatives such as for examples: caffeine, a minor stimulant, aminophylline, IBMX (3-isobutyl-1-methylxanthine), used as investigative tool in pharmacological research, paraxanthine, pentoxifylline, a drug that has the potential to enhance circulation and may have applicability in treatment of diabetes, fibrotic disorders, peripheral nerve damage, and microvascular injuries, theobromine and theophylline, a bronchodilator. Methylated xanthines act as both competitive nonselective phosphodiesterase inhibitors which raise intracellular cAMP, activate PKA, inhibit TNF-alpha and leukotriene synthesis, and reduce inflammation and innate immunity and nonselective adenosine receptor antagonists. Different analogues show varying potency at the numerous subtypes, and a wide range of synthetic xanthine derivatives (some nonmethylated) have been developed in the search for compounds with greater selectivity for phosphodiesterase enzyme or adenosine receptor subtypes.

PDE inhibitors include 1-(3-Chlorophenylamino)-4-phenylphthalazine and dipyridamol. Another PDE1 selective inhibitor is, for example, Vinpocetine.

PDE2 selective inhibitors include for example, EHNA (erythro-9-(2-hydroxy-3-nonyl)adenine) and Anagrelide.

PDE3 selective inhibitors include for example, sulmazole, ampozone, cilostamide, carbazeran piroximone, imazodan, siguazodan, adibendan, saterinone, emoradan, revizinone, and enoximone and milrinone. Some are used clinically for short-term treatment of cardiac failure. These drugs mimic sympathetic stimulation and increase cardiac output. PDE3 is sometimes referred to as cGMP-inhibited phosphodiesterase.

Examples of PDE3/4 inhibitors include benafentrine, trequinsin, zardaverine and tolafentrine.

PDE4 selective inhibitors include for example: winlcuder, denbufylline, rolipram, oxagrelate, nirtaquazone, motapizone, lixazinone, indolidan, olprinone, atizoram, dipamfylline, arofylline, filaminast, piclamilast, tibenelast, mopidamol, anagrelide, ibudilast, aminone, pimobendan, cilostazol, quazinone and N-(3,5-dichloropyrid-4-yl)-3-cyclopropylmethoxy-4-difluoromethoxybenzamide. Mesembrine, an alkaloid from the herb Sceletium tortuosum; Rolipram, used as investigative tool in pharmacological research; Ibudilast, a neuroprotective and bronchodilator drug used mainly in the treatment of asthma and stroke (inhibits PDE4 to the greatest extent, but also shows significant inhibition of other PDE subtypes, and so acts as a selective PDE4 inhibitor or a non-selective phosphodiesterase inhibitor, depending on the dose); Piclamilast, a more potent inhibitor than rolipram; Luteolin, supplement extracted from peanuts that also possesses IGF-1 properties; Drotaverine, used to alleviate renal colic pain, also to hasten cervical dilatation in labor, and Roflumilast, indicated for people with severe COPD to prevent symptoms such as coughing and excess mucus from worsening. PDE4 is the major cAMP-metabolizing enzyme found in inflammatory and immune cells. PDE4 inhibitors have proven potential as anti-inflammatory drugs, especially in inflammatory pulmonary diseases such as asthma, COPD, and rhinitis. They suppress the release of cytokines and other inflammatory signals, and inhibit the production of reactive oxygen species. PDE4 inhibitors may have antidepressive effects[26] and have also recently been proposed for use as antipsychotics.

PDE5 selective inhibitors include for example: Sildenafil, tadalafil, vardenafil, vesnarinone, zaprinast lodenafil, mirodenafil, udenafil and avanafil. PDE5, is cGMP-specific is responsible for the degradation of cGMP in the corpus cavernosum (these phosphodiesterase inhibitors are used primarily as remedies for erectile dysfunction, as well as having some other medical applications such as treatment of pulmonary hypertension); Dipyridamole (results in added benefit when given together with NO or statins); and newer and more-selective inhibitors are such as icariin, an active component of Epimedium grandiflorum, and possibly 4-Methylpiperazine and Pyrazolo Pyrimidin-7-1, components of the lichen Xanthoparmelia scabrosa.

PDE10 is selective inhibited by Papaverine, an opium alkaloid. PDE10A is almost exclusively expressed in the striatum and subsequent increase in cAMP and cGMP after PDE10A inhibition (e.g. by papaverine) is “a novel therapeutic avenue in the discovery of antipsychotics”.

Additional PDE inhibitors include those set forth in U.S. Pat. Nos. 8,153,104, 8,133,903, 8,114,419, 8,106,061, 8,084,261, 7,951,397, 7,897,633, 7,807,803, 7,795,378, 7,750,015, 7,737,155, 7,732,162, 7,723,342, 7,718,702, 7,671,070, 7,659,273, 7,605,138, 7,585,847, 7,576,066, 7,569,553, 7,563,790, 7,470,687, 7,396,814, 7,393,825, 7,375,100, 7,363,076, 7,304,086, 7,235,625, 7,153,824, 7,091,207, 7,056,936, 7,037,257, 7,022,709, 7,019,010, 6,992,070, 6,969,719, 6,964,780, 6,875,575, 6,743,799, 6,740,306, 6,716,830, 6,670,394, 6,642,244, 6,610,652, 6,555,547, 6,548,508, 6,541,487, 6,538,005, 6,534,519, 6,534,518, 6,479,505, 6,476,025, 6,436,971, 6,436,944, 6,428,478, 6,423,683, 6,399,579, 6,391,869, 6,380,196, 6,376,485, 6,333,354, 6,306,869, 6,303,789, 6,294,564, 6,288,118, 6,271,228, 6,235,782, 6,235,776, 6,225,315, 6,177,471, 6,143,757, 6,143,746, 6,127,378, 6,103,718, 6,080,790, 6,080,782, 6,077,854, 6,066,649, 6,060,501, 6,043,252, 6,011,037, 5,998,428, 5,962,492, 5,922,557, 5,902,824, 5,891,896, 5,874,437, 5,871,780, 5,866,593, 5,859,034, 5,849,770, 5,798,373, 5,786,354, 5,776,958, 5,712,298, 5,693,659, 5,681,961, 5,674,880, 5,622,977, 5,580,888, 5,491,147, 5,426,119, and 5,294,626, which are each incorporated herein by reference. Additional PDE2 inhibitors include those set forth in U.S. Pat. Nos. 6,555,547, 6,538,029, 6,479,493 and 6,465,494, which are each incorporated herein by reference. Additional PDE3 inhibitors include those set forth in U.S. Pat. Nos. 7,375,100, 7,056,936, 6,897,229, 6,716,871, 6,498,173, and 6,110,471, which are each incorporated herein by reference. Additional PDE4 inhibitors include those set forth in U.S. Pat. Nos. 8,153,646, 8,110,682, 8,030,340, 7,964,615, 7,960,433, 7,951,954, 7,902,224, 7,846,973, 7,759,353, 7,659,273, 7,557,247, 7,550,475, 7,550,464, 7,538,127, 7,517,889, 7,446,129, 7,439,393, 7,402,673, 7,375,100, 7,361,787, 7,253,189, 7,135,600, 7,101,866, 7,060,712, 7,056,936, 7,045,658, 6,953,774, 6,884,802, 6,858,596, 6,787,532, 6,747,043, 6,740,655, 6,713,509, 6,630,483, 6,436,971, 6,288,118, and 5,919,801, which are each incorporated herein by reference. Additional PDE5 inhibitors include those set forth in U.S. Pat. Nos. 7,449,462, 7,375,100, 6,969,507, 6,723,719, 6,677,335, 6,660,756, 6,538,029, 6,479,493, 6,476,078, 6,465,494, 6,451,807, 6,143,757, 6,143,746 and 6,043,252, which are each incorporated herein by reference. Additional PDE10 inhibitors include those set forth in U.S. Pat. No. 6,538,029 which is incorporated herein by reference.

### MRP Inhibitors

The human multidrug resistance proteins MRP4 and MRP5 are organic anion transporters that have the unusual ability to transport cyclic nucleotides including cGMP. Accordingly, cGMP levels may be increased by inhibition of MRP4 and MRP5. Compounds that inhibit MRP4 and MRP5 may include dipyridamole, dilazep, nitrobenzyl mercaptopurine riboside, sildenafil, trequinsin, zaprinast and MK571 (3-[[[3-[(1E)-2-(7-Chloro-2-quinolinyl)ethenyl]phenyl][[3-(dimethylamino)-3-oxopropyl]thio]methyl]thio]propanoic acid). These compounds may be more effective at inhibiting MRP4 than MRP5. Other compounds which may be useful as MRP inhibitors include sulfinpyrazone, zidovudine-monophosphate, genistein, indomethacin, and probenecid.

Cyclic GMP and/or cGMP Analogues

In some embodiments, the active agent comprises cyclic GMP. In some embodiments, the active agent comprises cGMP analogues such as for example 8-bromo-cGMP and 2-chloro-cGMP.

### Controlled Release Formulations

Controlled release compositions are provided for delivering to tissues of the duodenum, small intestine, large intestine, colon and/or rectum. The controlled release formulations comprise one or more active agents selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), Guanylyl cyclase C agonists, PDE Inhibitors, MRP inhibitors, cyclic GMP and cGMP analogues, wherein the active agents are formulated as a controlled release composition for controlled release to tissues of the duodenum, small intestine, large intestine, colon and/or rectum. Method of preventing GI syndrome in an individual undergoing chemotherapy or radiation therapy to treat cancer are provided which comprise the step of, prior to administration of chemotherapy or radiation to the individual, administering to the individual by oral administration an amount of the controlled release composition sufficient to elevate intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome. Methods of reducing gastrointestinal side effects in an individual undergoing chemotherapy or radiation therapy to treat cancer are provided which comprise the step of, prior to administration of chemotherapy or radiation to the individual, administering to the individual by oral administration an amount of the controlled release composition sufficient to elevate intracellular cGMP levels in gastrointestinal cells sufficient to arrest cell proliferation of said gastrointestinal cells and/or maintain genomic integrity by enhanced DNA damage sensing and repair for a period sufficient to increase survival of gastrointestinal cells and reduce severity of chemotherapy or radiation therapy side effects. Methods of treating an individual who has cancer are provided that comprise the steps of administering by oral administration to the individual the controlled release composition in an amount that elevates intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome; and administering to said individual chemotherapy or radiation an amount sufficient to treat cancer. Methods of treating an individual who has cancer are provided that comprise the steps of administering by oral administration to the individual the controlled release composition in an amount that elevates intracellular cGMP levels in gastrointestinal cells sufficient to arrest cell proliferation of said gastrointestinal cells and/or maintain genomic integrity by enhanced DNA damage sensing and repair for a period sufficient to increase survival of gastrointestinal cells and reduce severity of chemotherapy or radiation therapy side effects; and administering to said individual chemotherapy or radiation an amount sufficient to treat cancer. Methods of preventing GI syndrome in an individual who has been exposed to or who is at risk of exposure to sufficient doses of radiation to cause GI syndrome are provided that comprise the step of administering by oral administration to the individual who has been exposed to or who is at risk of exposure to sufficient doses of radiation to cause GI syndrome, an amount of the controlled release composition that elevates intracellular cGMP levels in gastrointestinal cells sufficient to prevent GI syndrome. Methods of treating an individual who has been exposed to a sufficient amount of radiation to cause radiation sickness are provided that comprise the step of administering to said individual by oral administration, an amount of the controlled release composition that elevates cGMP levels in gastrointestinal cells sufficient to elevate intracellular cGMP levels in gastrointestinal cells sufficient to arrest cell proliferation of said gastrointestinal cells and/or maintain genomic integrity by enhanced DNA damage sensing and repair for a period sufficient to reduce gastrointestinal damage. Methods of preventing side effects in an individual who is undergoing chemotherapy or radiation are provided that comprise the steps of administering to said individual by oral administration prior to administration of chemotherapy or radiation the controlled release composition that elevates cGMP levels in cells to be protected sufficient to arrest cell proliferation of said cells and/or maintain genomic integrity by enhanced DNA damage sensing and repair for a period sufficient to reduce damage to said cells. Methods of treating an individual who has cancer are provided that comprise the steps of administering to said individual an amount of the controlled release composition that elevates cGMP levels in cells to be protected sufficient to arrest cell proliferation of said cells and/or maintain genomic integrity by enhanced DNA damage sensing and repair for a period sufficient to reduce damage to said cells; and administering to said individual chemotherapy or radiation an amount sufficient to treat cancer.

In some embodiments, methods comprise delivery of one or more active agents selected from the group consisting of: Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that the release of the at least some if not the majority or all of the active agent bypasses the stomach and is delivered to tissues of the duodenum, small intestine, large intestine, colon and/or rectum. These formulations are particularly useful in those cases in which the active agent is either inactivated by the stomach or taken up by the stomach, in either case thereby preventing the active agent from reaching the tissue downstream of the stomach where activity is desirable. In some embodiments, the preferred site of release the duodenum. In some embodiments, the preferred site of release the small intestine. In some embodiments, the preferred site of release the large intestine. In some embodiments, the preferred site of release the colon. Bypassing the stomach and releasing the drug after it has passed through the stomach ensures tissue specific delivery of active agent in effective amounts.

The methods provide more effective delivery of active agents to colorectal track including the duodenum, the small and large intestines and the colon. Formulations are provided to deliver active agent throughout the colorectal track or to specific tissue within in.

Some embodiments utilize GCC Agonists, Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors and/or cyclic GMP and/or cGMP analogues and/or PDE inhibitors formulated from controlled release whereby the release of the at least some if not the majority or all of the active agent bypasses the stomach and is delivered to tissues of the duodenum, small intestine, large intestine, colon and/or rectum. These formulations are particularly useful in those cases in which the active agent is either inactivated by the stomach or taken up by the stomach, in either case thereby preventing the active agent from reaching the tissue downstream of the stomach where activity is desirable. In some embodiments, the preferred site of release the duodenum. In some embodiments, the preferred site of release the small intestine. In some embodiments, the preferred site of release the large intestine. In some embodiments, the preferred site of release the colon.

Most enteric coatings are intended to protect contents from stomach acid. Accordingly, they are designed to release active agent upon passing through the stomach. The coatings and encapsulations used herein are provided to release active agents upon passing the colorectal track. This can be accomplished in several ways.

Enteric formulations are described in U.S. Pat. No. 4,601,896, U.S. Pat. No. 4,729,893, U.S. Pat. No. 4,849,227, U.S. Pat. No. 5,271,961, U.S. Pat. No. 5,350,741, and U.S. Pat. No. 5,399,347. Oral and rectal formulations are taught in Remington's Pharmaceutical Sciences, 18th Edition, 1990, Mack Publishing Co., Easton Pa. which is incorporated herein by reference.

According to some embodiments, active agents are coated or encapsulated with a sufficient amount of coating material that the time required for the coating material to dissolve and release the active agents corresponds with the time required for the coated or encapsulated composition to travel from the mouth to the colorectal track.

According to some embodiments, the active agents are coated or encapsulated with coating material that does not fully dissolve and release the active agents until it comes in contact with conditions present in the colorectal track. Such conditions may include the presence of enzymes in the colorectal track, pH, tonicity, or other conditions that vary relative to the small intestine.

According to some embodiments, the active agents are coated or encapsulated with coating material that is designed to dissolve in stages as it passes from stomach to small intestine to large intestine. The active agents are released upon dissolution of the final stage which occurs in the colorectal track.

In some embodiments, the formulations are provided for release of active agent in specific tissues or regions of the colorectal track, for example, the duodenum, the small intestine, the large intestine or the colon.

Examples of technologies which may be used to formulate active agents for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. No. 6,309,666; U.S. Pat. No. 6,569,463, U.S. Pat. No. 6,214,378; U.S. Pat. No. 6,248,363; U.S. Pat. No. 6,458,383, U.S. Pat. No. 6,531,152, U.S. Pat. No. 5,576,020, U.S. Pat. No. 5,654,004, U.S. Pat. No. 5,294,448, U.S. Pat. No. 6,309,663, U.S. Pat. No. 5,525,634, U.S. Pat. No. 6,248,362, U.S. Pat. No. 5,843,479, and U.S. Pat. No. 5,614,220, which are each incorporated herein by reference.

Controlled release formulations are well known including those which are particularly suited for release of active agent into the duodenum. Examples of controlled release formulations which may be used include U.S. Patent Application Publication 2010/0278912, U.S. Pat. No. 4,792,452, U.S. Patent Application Publication 2005/0080137, U.S. Patent Application Publication 2006/0159760, U.S. Patent Application Publication 2011/0251231, U.S. Pat. No. 5,443,843, U.S. Patent Application Publication 2008/0153779, U.S. Patent Application Publication 2009/0191282, U.S. Patent Application Publication 2003/0228362, U.S. Patent Application Publication 2004/0224019, U.S. Patent Application Publication 2010/0129442, U.S. Patent Application Publication 2007/0148153, U.S. Pat. No. 5,536,507, U.S. Pat. No. 7,790,755, U.S. Patent Application Publication 2005/0058704, U.S. Patent Application Publication 2001/0026800, U.S. Patent Application Publication 2009/0175939, US 2002/0192285, U.S. Patent Application Publication 2008/0145417, U.S. Patent Application Publication 2009/0053308, U.S. Pat. No. 8,043,630, U.S. Patent Application Publication 2011/0053866, U.S. Patent Application Publication 2009/0142378, U.S. Patent Application Publication 2006/0099256, U.S. Patent Application Publication 2009/0104264, U.S. Patent Application Publication 2004/0052846, U.S. Patent Application Publication 2004/0053817, U.S. Pat. No. 4,013,784, U.S. Pat. No. 5,693,340, U.S. Patent Application Publication 2011/0159093, U.S. Patent Application Publication 2009/0214640, U.S. Pat. No. 5,133,974, U.S. Pat. No. 5,026,559, U.S. Patent Application Publication 2010/0166864, U.S. Patent Application Publication 2002/0110595, U.S. Patent Application Publication 2007/0148153, U.S. Patent Application Publication 2009/0220611, U.S. Patent Application Publication 2010/0255087 and U.S. Patent Application Publication 2009/0042889, each of which is incorporated herein by reference. Other examples of technologies which may be used to formulate active agents for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

### Patient Populations

Prior to receiving anticancer chemotherapy or radiation, patients undergoing chemotherapy and/or radiation therapy may be provided with compositions which elevate cGMP levels in non-cancer tissues that comprise dividing cells such as gastrointestinal tissue in order to protect those tissues from deleterious side effects brought on by non-specific toxicity against dividing cells. Elevated levels of cGMP are maintained during the period of time chemotherapeutics and/or radiation is a present. By elevating cGMP levels in non-cancer cells, individual patients will experience reduced toxicity and side effects which often accompany chemotherapy and radiation. Higher doses of chemotherapy and radiation may be tolerated because of reduced side effects to non-cancer cells.

Individuals undergoing radiation therapy or treatment with one or more of chemotherapeutic drugs such as alkylating agents, antimetabolites, anthracyclines, plant alkaloids, topoisomerase inhibitors, and other antitumour agents which affect cell division or DNA synthesis and function in some way will typically benefit from protection of normally dividing non-cancer cells because the radiation and chemotherapy is not selective and will effect normally dividing non-cancer cells and well as cancer cells.

### Toxic Chemotherapy

Alkylating agents are classified under LOlA in the Anatomical Therapeutic Chemical Classification System. These agents function as anticancer agents by damaging DNA through their attachment to the alkyl group attached to the guanine base of DNA, at the number 7 nitrogen atom of the imidazole ring. Alkylating agents are toxic to normal cells and can cause severe side effects when used as anticancer agents. Classical alkylating agents include true alkyl groups, include the Nitrogen mustards such as Cyclophosphamide, Mechlorethamine or mustine (HN2), Uramustine or uracil mustard, Melphalan, Chlorambucil, Ifosfamidel the Nitrosoureas such as Carmustine, Lomustine, Streptozocin; and the Alkyl sulfonates such as Busulfan. Thiotepa and its analogues are often but not always considered classical. Alkylating-like Platinum-based chemotherapeutic drugs, sometimes referred to as platinum analogs, do not have an alkyl group, but nevertheless damage DNA. These compounds are sometimes described as “alkylating-like” because they coordinate to DNA to interfere with DNA repair. These agents also bind at N7 of guanine Examples of Alkylating-like Platinum-based chemotherapeutic drugs include Cisplatin, Carboplatin, Nedaplatin, Oxaliplatin, Satraplatin, Triplatin, and tetranitrate. While the platinum agents are sometimes described as nonclassical, more typically, the nonclassical alkylating agents include procarbazine and altretamine. Tetrazines (dacarbazine, mitozolomide, temozolomide) are sometimes also listed in this category.

Antimetabolite agents are classified under L01B in the ATC system. They are toxic chemicals that inhibit the use of a metabolite that is part of normal metabolism, thus halting cell growth and cell division by interfering with DNA production and therefore cell division and the growth of tumors. Antimetabolite agents are toxic to normal dividing cells as well as cancer cells and can cause severe side effects when used as anticancer agents. Anti-metabolites include purine analogs such as azathioprine, mercaptopurine, thioguanine, fludarabine, pentostatin and cladribine; pyrimidine analogs such as 5-fluorouracil (5FU) a thymidylate synthase inhibitor, floxuridine, cytosine arabinoside (Cytarabine), and antifolates such as methotrexate, trimethoprim, pyrimethamine, pemetrexed, raltitrexed and pralatrexate.

Anthracyclines are a class of anti-cancer drugs derived from Streptomyces bacteria. Anthracycline mechanisms of action include inhibition of DNA and RNA synthesis by intercalating between base pairs of the DNA/RNA strand, and thus preventing the replication of rapidly-growing cancer cells; inhibition of topoiosomerase II enzyme, preventing the relaxing of supercoiled DNA and thus blocking DNA transcription and replication, and creation of iron-mediated free oxygen radicals that damage the DNA and cell membranes. Examples of anthracyclines include daunorubicin (Daunomycin), liposomal daunorubicin, doxorubicin (Adriamycin), liposomal doxorubicin, epirubicin, idarubicin, valrubicin, and the anthracycline analog mitoxantrone.

Alkaloids which block cell division by preventing microtubule function are useful as anticancer agents. Since microtubules are necessary for cell division, preventing their formation prevents cell division from occurring. Vinca alkaloids, which are classified under L01CA in the ATC system, bind to tubulin, and inhibit assembly of microtubules during the M phase of the cell cycle. The vinca alkaloids include vincristine, vinblastine, vinorelbine and vindesine. Colcemid and nocodazole, which are similar to vinca alkaloids, are anti-mitotic and anti-microtubule agents. drugs. Podophyllotoxin, which is classified under L01CB in the ATC system, is a plant-derived compound which is used to produce two other cytostatic drugs, etoposide and teniposide that prevent the cell from entering the G1 phase (the start of DNA replication) and the S phase (the replication of DNA). Taxanes which is classified under L01CD in the ATC system, include taxane or paclitaxel (Taxol). Docetaxel is a semi-synthetic analogue of paclitaxel. Taxanes enhance stability of microtubules, preventing the separation of chromosomes during anaphase.

Some topoisomerase inhibitors are classified under L01CB in the ATC system which inhibit the topoisomerase enzymes that play essential rolls in maintaining DNA supercoiling. By upsetting proper DNA supercoiling, inhibition of either or the type I or type II topoisomerases interferes with both transcription and replication of DNA. Examples of type I topoisomerase inhibitors include camptothecins: irinotecan and topotecan. Examples of type II inhibitors include amsacrine, etoposide, etoposide phosphate, and teniposide which are semisynthetic derivatives of naturally occurring alkaloids, epipodophyllotoxins.

Other antineoplastic compounds function by generating free radicals. Examples include cytotoxic antibiotics such as bleomycin (L01DC01), plicamycin (L01DC02) and mitomycin (L01DC03).

### Toxic Radiation

Radiation therapy uses photons or charged particle to damage the DNA of cancerous cells. The damage may be direct or indirect ionizing the atoms which make up the DNA chain. Indirect ionization happens as a result of the ionization of water, forming free radicals, notably hydroxyl radicals, which then damage the DNA. Direct damage to DNA occurs through high-LET (linear energy transfer) charged particles such as proton, boron, carbon or neon ions which have an antitumor effect which is independent of tumor oxygen supply because these particles act mostly via direct energy transfer usually causing double-stranded DNA breaks. Conventional external beam radiotherapy is delivered via two-dimensional beams using linear accelerator machines. Stereotactic Radiation is a specialized type of external beam radiation therapy that uses focused radiation beams targeting a well-defined tumor using extremely detailed imaging scans.

In addition to radiation used in radiotherapy, GI syndrome and radiation sickness can occur when an individual is unintentionally exposed to large amounts of radiation such as the result of an accident or deliberate release of radioactive material. In such events, GI syndrome and radiation sickness can be prevented by administering compounds that elevate cGMP levels in gastrointestinal cells sufficient to elevate intracellular cGMP levels in gastrointestinal cells sufficient to arrest cell proliferation of gastrointestinal cells and/or maintain genomic integrity by enhanced DNA damage sensing and repair for a period sufficient to reduce damage to gastrointestinal cells and prevent GUI syndrome and/or radiation sickness. In some embodiments, the compounds that elevate cGMP levels may be administered starting immediately following exposure to radiation or, if in the case of emergency workers, prior to entering an area of high levels of radiation. In some embodiments, the compounds that elevate cGMP levels may be administered to individuals who are experiencing symptoms of radiation sickness.

### Protection of Normal-Dividing Non-Cancer Intestinal Cells

Protection of normally dividing non-cancer intestinal cells can be achieved by elevation of cGMP levels. The elevation of cGMP levels in normally dividing non-cancer intestinal cells may be achieved by administration of one or more compounds in amounts sufficient to achieve elevated cGMP levels. The one or more compounds are delivered to intestinal cells in amounts and frequency sufficient to sustain the cGMP at elevated levels prior to and during exposure to toxic chemotherapy and/or radiation.

In some embodiments, compounds which elevate cGMP do so through interaction with a cellular receptor present on the cells. GCC agonists may be delivered by routes that provide the agonist to contact the GCC expressed by intestinal cells in order to activate the receptors. In some embodiments, the compounds which elevate cGMP levels may be taken up by cell by other means. For example, cells which contain specific PDE or MRP isoforms would indicate the inhibitory compounds used. For example, cells expressing PDE5 would be protected by use of PDE5 inhibitors while cells expressing MRP5 would be protected by use of MRP5 inhibitors. In such embodiments, the compounds may be administered by any route such that they can be taken up by cells.

Regardless of the mechanism for delivery to the cell, the dose and route of delivery preferably minimizes uptake by cancer cells if the cancer cells are the type which are protected by elevated cGMP levels and if the compound used can affect such cells. In embodiments in which cGMP levels are to be increased in normal intestinal cells using GCC agonists, oral delivery to the gut is preferred. Compounds must be protected from degradation or uptake prior to reaching the gut. Many known peptide agonists of GCC are stable in the acidic environment of the stomach and will survive in active form when passing through the stomach to the gut. Some compounds may require enteric coating. In the case of GCC expression in cell lining the gut, the delivery of GCC agonist through local delivery directly to the interior of the intestinal, by oral or rectal administration for example, is particularly useful in that cells outside the gut will not be exposed to the GCC agonist since the tight junctions of intestinal tissue prevent direct passage of most GCC agonists.

The amount and duration of delivery of compounds which elevate cGMP levels in dividing, non-cancer intestinal cells is sufficient to maintain levels elevated to protective levels prior to and during exposure to toxic chemotherapy and radiation. The result will be the protection of a sufficient number of such cells through p53 mediated cell survival to effectively reduce the severity of side effects and/or allow for higher levels of chemotherapy and radiation to be used without being lethal or causing undesirable or intolerable levels of side effects.

In some embodiments the one or more compounds which increase cGMP levels is formulated as a injectable pharmaceutical composition suitable for parenteral administration such as by intravenous, intraarterial, intramuscular, intradermal or subcutaneous injection. Accordingly, the composition is a sterile, pyrogen-free preparation that has the structural/physical characteristics required for injectable products; i.e. it meets well known standards recognized by those skilled in the art for purity, pH, isotonicity, sterility, and particulate matter.

In some preferred embodiments, the one or more compounds which increase cGMP levels is administered orally or rectally and the compositions is formulated as pharmaceutical composition suitable for oral or rectal administration. Some embodiments providing the one or more compounds which increase cGMP levels are provided as suitable for oral administration and formulated for sustained release. Some embodiments providing the one or more compounds which increase cGMP levels are provided as suitable for oral administration and formulated by enteric coating to release the active agent in the intestine. Enteric formulations are described in U.S. Pat. No. 4,601,896, U.S. Pat. No. 4,729,893, U.S. Pat. No. 4,849,227, U.S. Pat. No. 5,271,961, U.S. Pat. No. 5,350,741, and U.S. Pat. No. 5,399,347. Oral and rectal formulation are taught in Remington's Pharmaceutical Sciences, 18th Edition, 1990, Mack Publishing Co., Easton Pa. which is incorporated herein by reference.

Alternative embodiments include sustained release formulations and implant devices which provide continuous delivery of the one or more compounds which increase cGMP levels. In some embodiments, the one or more compounds which increase cGMP levels is administered topically, intrathecally, intraventricularly, intrapleurally, intrabronchially, or intracranially.

Generally, the one or more compounds which increase cGMP levels must be present at a sufficient level for a sustained amount of time to increase cGMP levels during the period the cells are potentially exposed to toxic chemotherapy or radiation. Generally, enough of the one or more compounds which increase cGMP levels must be administered initially and/or by continuous administration to maintain the concentration of sufficient to maintain elevated cGMP levels for most if not the entire period of time the patient is exposed to toxic chemotherapy or radiation. It is preferred that elevated cGMP levels sufficient to enhance p53 mediated cell survival be maintained for at least about 6 hours, preferably about for at least about 8 hours, more preferably about for at least about 12 hours, in some embodiments at least 16 hours, in some embodiments at least 20 hours, in some embodiments at least 24 hours, in some embodiments at least 36 hours, in some embodiments at least 48 hours, in some embodiments at least 72 hours, in some embodiments at least 96 hours, in some embodiments at least one week, in some embodiments at least two weeks, in some embodiments at least three weeks and up to about 4 weeks or more. It is important that the dosage and administration be sufficient for the cGMP level to be elevated in an amount sufficient for sufficient time to enhance p53 mediated cell survival such that the severity of side effects is reduced and/or the tolerable dose of chemotherapeutic or radiation can be increased. Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

In some embodiments, a GCC agonist such as a peptide having SEQ ID NO:2, 3 or 5-58 is administered to the individual. In practicing the method, the compounds may be administered singly or in combination with other compounds. In the method, the compounds are preferably administered with a pharmaceutically acceptable carrier selected on the basis of the selected route of administration and standard pharmaceutical practice. It is contemplated that the daily dosage of a compound used in the method will be in the range of from about 1 micrograms to about 10 grams per day. In some preferred embodiments, the daily dosage compound will be in the range of from about 10 mg to about 1 gram per day. In some preferred embodiments, the daily dosage compound will be in the range of from about 100 mg to about 500 mg per day. It is contemplated that the daily dosage of a compound used in the method that is the invention will be in the range of from about 1 μg to about 100 mg per kg of body weight, in some embodiments, from about 1 μg to about 40 mg per kg body weight; in some embodiments from about 10 μg to about 20 mg per kg per day, and in some embodiments 10 μg to about 1 mg per kg per day. Pharmaceutical compositions may be administered in a single dosage, divided dosages or in sustained release. In some preferred embodiments, the compound will be administered in multiple doses per day. In some preferred embodiments, the compound will be administered in 3-4 doses per day. The method of administering compounds include administration as a pharmaceutical composition orally in solid dosage forms, such as capsules, tablets, and powders, or in liquid dosage forms, such as elixirs, syrups, and suspensions. Compounds may be mixed with powdered carriers, such as lactose, sucrose, mannitol, starch, cellulose derivatives, magnesium stearate, and stearic acid for insertion into gelatin capsules, or for forming into tablets. Both tablets and capsules may be manufactured as sustained release products for continuous release of medication over a period of hours. Compressed tablets can be sugar or film coated to mask any unpleasant taste and protect the tablet from the atmosphere or enteric coated for selective disintegration in the gastrointestinal tract. In some preferred embodiments, compounds are delivered orally and are coated with an enteric coating which makes the compounds available upon passing through the stomach and entering the intestinal tract, preferably upon entering the large intestine. U.S. Pat. No. 4,079,125, which is incorporated herein by reference, teaches enteric coating which may be used to prepare enteric coated compound of the inventions useful in the methods of the invention. Liquid dosage forms for oral administration may contain coloring and flavoring to increase patient acceptance, in addition to a pharmaceutically acceptable diluent such as water, buffer or saline solution. For parenteral administration, a compound may be mixed with a suitable carrier or diluent such as water, a oil, saline solution, aqueous dextrose (glucose), and related sugar solutions, and glycols such as propylene glycol or polyethylene glycols. Solutions for parenteral administration contain preferably a water soluble salt of the compound. Stabilizing agents, antioxidizing agents and preservatives may also be added. Suitable antioxidizing agents include sodium bisulfite, sodium sulfite, and ascorbic acid, citric acid and its salts, and sodium EDTA. Suitable preservatives include benzalkonium chloride, methyl- or propyl-paraben, and chlorbutanol.

### Sensitizing Activity in Some Cancers

As noted above, cGMP promotes cell death in response to DNA damage by chemotherapy or radiation therapy in a variety of cancer cells including lung, breast, prostate, colorectal, and liver cancer cells. In view of the tissue specific effect of cGMP on cell death in the intestine, increase in cGMP in intestinal cells in conjunction with chemotherapy or radiation therapy to reduce GI side effects and in some cases may potentiate the therapeutic efficacy for lung, breast, prostate, colorectal, and liver cancers.

In the treatment of cancer of a type which is rendered more susceptible to chemotherapy- or radiotherapy-induced cell death when cGMP levels are elevated, compounds which elevate cGMP may be administered in doses and by routes of administration a manner which delivered sufficient compound to cancer cells to increase the effectiveness of chemotherapy and radiotherapy to kill the cancer cells. In some embodiments, the compounds may potentiate chemotherapy- or radiotherapy-induced cell death in cancer cells while protecting non-cancer cells from chemotherapy or radiation therapy through p53 mediated cell survival.

### Other Cell Types

In some embodiments, the normal non-dividing cells may be other types of cells for which elevated cGMP can enhance p53 mediated cell survival. In some embodiments, the normal non-dividing cells may be hair follicles, skin, lungs, nasal passages, other mucosae or tissue in the oral cavity. Compounds may be delivered topically to the scalp or to tissue of the oral cavity including mouth, tongue, gums, and buccal tissue, preferably formulated for local uptake with minimal system uptake. Compounds may be delivered using an inhalation device and/or nasal spray, preferably formulated for local uptake with minimal system uptake. Similarly, compounds which elevate cGMP levels in normal dividing non-cancer cells such as other cells of the mucosae or such as skin cells may be formulated for preferential uptake and delivered directly to such cells. Such delivery may include intraocularly, intravaginally, intraurethraly, rectal/anal or topically.

The amount and duration of delivery of compounds which elevate cGMP levels in dividing, non-cancer cells which can be protected by p53 mediated cell survival by elevated cGMP is sufficient to maintain levels elevated to protective levels prior to and during exposure to toxic chemotherapy and radiation. The result will be the protection of a sufficient number of such cells by p53 mediated cell survival to effectively reduce the severity of side effects and/or allow for higher levels of chemotherapy and radiation to be used without being lethal or causing undesirable or intolerable levels of side effects.

### Example

Experiments were performed to show the requirement of GCC activation in the protection of small intestine and colon cells from apoptosis and genotoxic induced cell death. The role of activation of GCC and of the presence of p53 was also evaluated. Test on cancer cells were performed to compare the protection by cGMP of colon cancer cells from cell death in compared to cGMP's potentiation of cell death in other cancers.

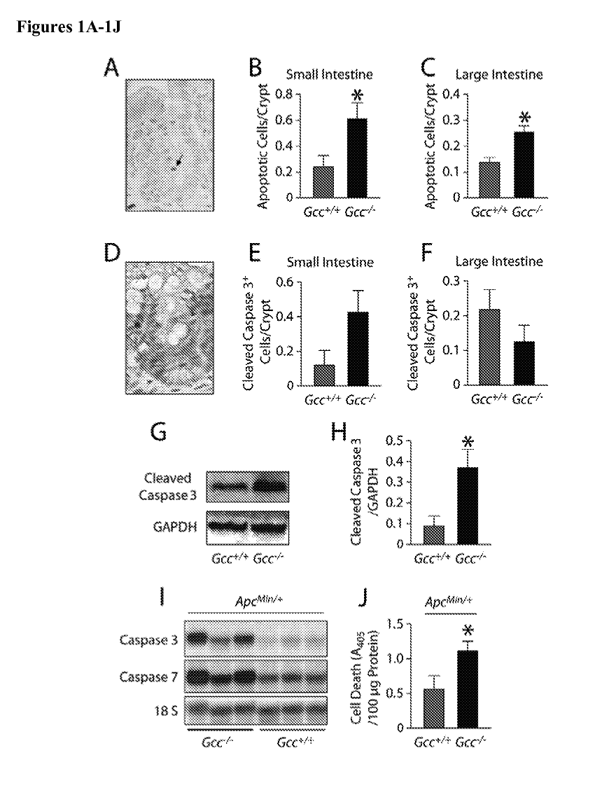

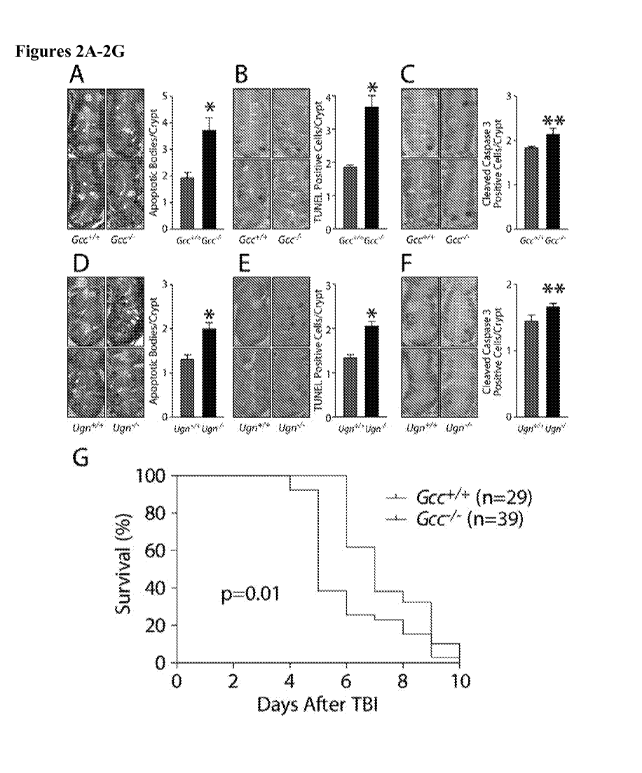

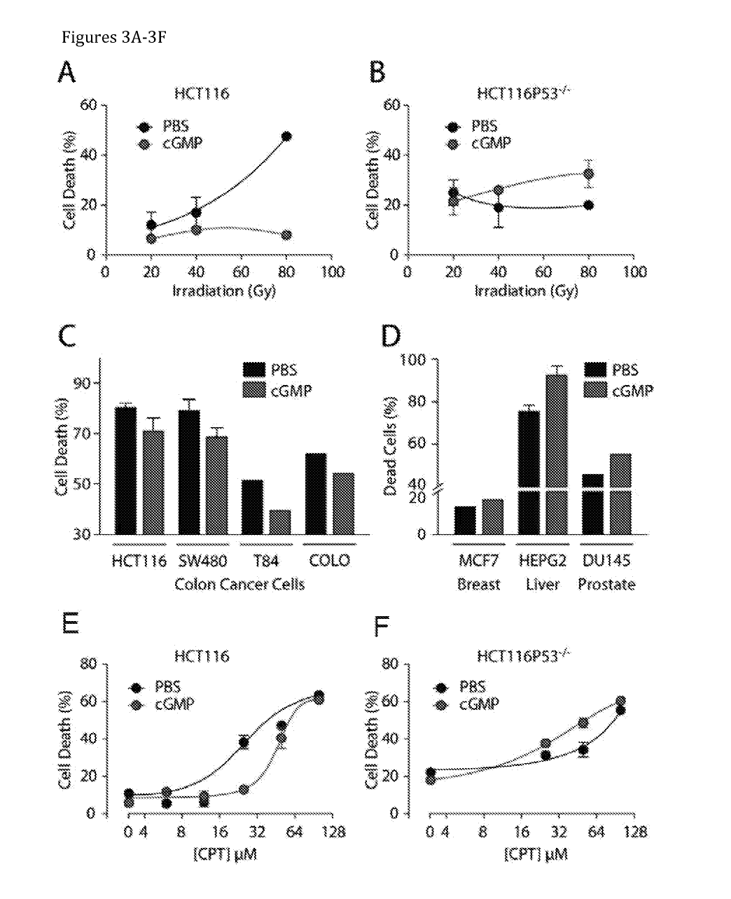

The data show that GCC-cGMP axis protects cell death in intestinal epithelium in physiological conditions (FIG. 1A-H) and in response to genotoxic insults (FIG. 1-J and FIG. 2). Eliminating the GCC-cGMP axis in mice, including either the receptor GCC or an endogenous ligand uroguanylin, increases radiation-induced intestinal crypt cell apoptosis (FIG. 2A-F). Elimination of GCC increased lethal GI toxicity induced by ionizing radiation (IR) in Gcc^{-/-}, compared to Gcc^{+/+}, mice (FIG. 2G). Conversely, GCC downstream signaling in human colon cells in vitro attenuates chemo- and radiation-induced cell death in a p53-dependent fashion (FIG. 3A, B, E, F). Moreover, cGMP promotes cell death in human breast, liver and prostate cancer cells induced by chemo-toxicity (FIG. 3 C, D). These observations suggest that the GCC-cGMP axis maintains genomic and physical barrier integrity in intestine by suppressing cell death following exposure to chemotherapy or ionizing radiation, protecting against chemo- or lethal radiation-induced GI toxicity. They underscore the potential of oral administration of GCC ligands for targeted chemo- and radio-protection of intestinal epithelia to prevent GI toxicity to improve the management of cancer therapy.

FIGS. 1A-J show that the GCC-cGMP axis protects cell death in intestinal epithelium in physiological conditions. One hundred to eight hundred crypts/intestinal segment from 3-5 Gcc^{+/+} and Gcc^{-/-} mice were scored for apoptosis and the presence of apoptosis related markers. Knockout mice which do not express GCC showed an increased apoptosis in small intestine and colon, quantified by TUNEL staining (A, B and C) as well as by detection of cleaved-caspase 3 staining (D, E and F). Apoptosis was also quantified by immunoblot to cleaved caspase 3 in the intestinal mucosa from 3 Gcc^{+/+} and Gcc^{-/-} mice (G, H) (Li P, Lin J E, Chervoneva I, Schulz S, Waldman S A, Pitari G M: Homeostatic control of the crypt-villus axis by the bacterial enterotoxin receptor guanylyl cyclase C restricts the proliferating compartment in intestine, Am J Pathol 2007, 171:1847-1858). Similarly, elimination of GCC increased mRNA expression of caspase 3 and 7 in Apc^{Min/+} mice (I), and apoptotic protein level was quantified by ELISA in 5 Apc^{Min/+}Gcc^{+/+} and 6 Apc^{Min/+}Gcc^{-/-} mice (J) (Mann E A, Steinbrecher K A, Stroup C, Witte D P, Cohen M B, Giannella R A: Lack of guanylyl cyclase C, the receptor for Escherichia coli heat-stable enterotoxin, results in reduced polyp formation and increased apoptosis in the multiple intestinal neoplasia (Min) mouse model, Int J Cancer 2005, 116:500-505). *, p<0.05

FIGS. 2A-G show GCC signaling prevents intestinal cell death induced by IR and protects mouse death from total body irradiation (TBI)-induced GI toxicity. Elimination of both GCC (A, B and C) and uroguanylin (D, E and F) in mice increased cell death in small intestine in response to 5 Gy γ-irradiation, quantified by H&E (A, D), TUNEL (B, E) and cleaved caspase 3 staining (C, F) in 6-11 Gcc^{+/+} and Gcc^{-/-} mice (Garin-Laflam M P, Steinbrecher K A, Rudolph J A, Mao J, Cohen M B: Activation of guanylate cyclase C signaling pathway protects intestinal epithelial cells from acute radiation-induced apoptosis, Am J Physiol Gastrointest Liver Physiol 2009, 296:G740-749). Gcc^{-/-} mice exhibited accelerated death reflecting TBI-induced GI toxicity compared to Gcc^{+/+} mice (median survival: 7 d, Gcc mice; 5 d, Gcc^{-/-} mice). The hazard ratio for death in Gcc^{-/-} mice was 2.17 (95% confidence interval: 1.17-4.01, p=0.01 by the log-rank test). *, p<0.05; **, p<0.01

FIGS. 3A-F show that cGMP protects cell death in human intestinal epithelial cells in response to genotoxic insults, while potentiates cell death in human breast, liver and prostate cancer cells. HCT116 cells (P53 wildtype) preconditioned with cGMP resisted IR-induced cell death (A) and the protection by cGMP preconditioning did not occur in HCT116P53^{-/-} cells (P53 null) (B). cGMP protected cell death in human colon cells in response to irinotecan (CPT11, 100 μM) challenge (D), while cGMP precondition promoted cell death in human breast, liver and prostate cancer cells induced by CPT11 (E). Similarly, HCT116 cells preconditioned with cGMP resisted CPT11-induced cell death (F) and the protection was P53 dependent (G).

1. The method of claim 29 wherein the individual undergoing chemotherapy or radiation therapy to treat cancer.

2. The method of claim 31 wherein the individual undergoing chemotherapy or radiation therapy to treat cancer, the side effects are gastrointestinal side effects, and the cells to be protected are gastrointestinal cells, and wherein said amount of one or more compounds elevates intracellular cGMP levels in gastrointestinal cells sufficient to arrest cell proliferation of said gastrointestinal cells and/or maintain genomic integrity by enhanced DNA damage sensing and repair for a period sufficient to increase survival of gastrointestinal cells and reduce severity of chemotherapy or radiation therapy side effects.

3. The method of claim 32, wherein the cells to be protected are gastrointestinal cells and wherein said amount of one or more compounds elevates intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome.

4. The method of claim 3 further comprising the step of subsequently administering to said individual chemotherapy or radiation an amount sufficient to treat cancer.

5. (canceled)

6. The method of claim 29 comprising administering to said individual a GCC agonist selected from the group consisting of SEQ ID NOs:2, 3 and 5-58.

7. (canceled)

8. The method of claim 29 comprising administering to said individual a PDE5 inhibitor.

9. (canceled)

10. The method of claim 29 comprising administering to said individual by oral administration a controlled release composition comprising one or more active agents selected from the group consisting of: Guanylyl cyclase C (GCC) agonists, Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that active agent is released and delivered to duodenum, small intestine, large intestine, colon and/or rectum tissue.

11. The method of claim 2 wherein one or more compounds that elevates cGMP levels is administered to said individual at a time selected from the group consisting of: 24 hours prior to administering to said individual chemotherapy or radiation an amount sufficient to treat cancer; 48 hours prior to administering to said individual chemotherapy or radiation an amount sufficient to treat cancer; 72 hours prior to administering to said individual chemotherapy or radiation an amount sufficient to treat cancer; and 96 hours prior to administering to said individual chemotherapy or radiation an amount sufficient to treat cancer.

12-18. (canceled)

19. The method of claim 2 wherein said individual is administered radiation therapy and/or chemotherapy.

20-28. (canceled)

29. A method of preventing GI syndrome in an individual who has been exposed to or who is at risk of exposure to sufficient doses of radiation to cause GI syndrome comprising the step of:
administering to an individual identified as an individual who has been exposed to or who is at risk of exposure to sufficient doses of radiation to cause GI syndrome, an amount of one or more compounds that elevates cGMP levels in gastrointestinal cells sufficient to prevent GI syndrome.

30. A method of treating an individual who has been exposed to a sufficient amount of radiation to cause radiation sickness comprising the step of:
administering to said individual, an amount of one or more compounds that elevates cGMP levels in gastrointestinal cells sufficient to elevate intracellular cGMP levels in gastrointestinal cells sufficient to arrest cell proliferation of said gastrointestinal cells and/or maintain genomic integrity by enhanced DNA damage sensing and repair for a period sufficient to reduce gastrointestinal damage.

31. A method of reducing or preventing side effects in an individual who is undergoing chemotherapy or radiation comprising the steps of
administering to said individual prior to administration of chemotherapy or radiation an amount of one or more compounds that elevates cGMP levels in cells to be protected sufficient to arrest cell proliferation of said cells and/or maintain genomic integrity by enhanced DNA damage sensing and repair for a period sufficient to reduce damage to said cells.

32. A method of treating an individual who has cancer comprising the steps of
administering to said individual an amount of one or more compounds that elevates cGMP levels in cells to be protected sufficient to arrest cell proliferation of said cells and/or maintain genomic integrity by enhanced DNA damage sensing and repair for a period sufficient to reduce damage to said cells; and
administering to said individual chemotherapy or radiation an amount sufficient to treat cancer.

33. The method of claim 31 comprising administering to said individual one or more compounds selected from the group consisting of a GCA agonist, a GCB agonist, a GCC agonist, a soluble guanylyl cyclase activator, a PDE inhibitor, a MRP4 inhibitor and a MRP5 inhibitor.

34. The method of claim 31 comprising administering to said individual ANP and/or BNP or CNP.

35. (canceled)

36. The method of claim 31 comprising administering to said individual: one or more soluble guanylyl cyclase activators selected from the group consisting of nitric oxide, nitrovasodilators, protoprophyrin IX, and direct activators.

37. (canceled)

38. The method of claim 31 comprising administering to said individual one or more PDE5 inhibitors.

39. (canceled)

40. The method of claim 31 comprising administering to said individual by oral administration a controlled release composition comprising one or more active agents selected from the group consisting of: Guanylyl cyclase C (GCC) agonists, Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that active agent is released and delivered to duodenum, small intestine, large intestine, colon and/or rectum tissue.

41. The method of claim of claim 31 wherein the compound that elevates cGMP levels is administered to said individual at a time selected from the group of: 24 hours prior to administering to said individual chemotherapy or radiation an amount sufficient to treat cancer; 48 hours prior to administering to said individual chemotherapy or radiation an amount sufficient to treat cancer; 72 hours prior to administering to said individual chemotherapy or radiation an amount sufficient to treat cancer; and 96 hours prior to administering to said individual chemotherapy or radiation an amount sufficient to treat cancer.

42-44. (canceled)

45. The method of claim 31 wherein the compound that elevates cGMP is administered in multiple doses.

46. (canceled)
